(19) United States
(12) Patent Application Publication
(10) Pub. No.: US 2023/0329853 A1
(43) Pub. Date: Oct. 19, 2023

(54) INTERVENTRICULAR SEPTUM PROTECTION

(71) Applicant: SV SWISSVORTEX AG, Zürich (CH)

(72) Inventors: Andrea GUIDOTTI, Zurich (CH); Idan TOBIS, Beit Hashmonai (IL); David ZARBATANY, Laguna Niguel, CA (US); Pietro GOZZOLI, Zurich (CH); Ricardo ROMAN, Chula Vista, CA (US)

(73) Assignee: SV SWISSVORTEX AG, Zürich (CH)

(21) Appl. No.: 18/043,197

(22) PCT Filed: Aug. 26, 2021
(86) PCT No.: PCT/IB2021/057816

§ 371 Date: Feb. 27, 2023

Related U.S. Application Data
(60) Provisional application No. 63/070,300, filed on Aug. 26, 2020.

(51) Int. Cl. A61F 2/01; A61F 2/24

(52) U.S. Cl. CPC A61F 2/013; A61F 2/2427; A61F 2/011; A61F 2/0105; A61F 2002/018; A61F 2002/016; A61M 25/0068

(57) ABSTRACT

A valve-delivery-assist tool (20, 120, 220, 320) is provided for use during a transcatheter aortic valve implantation (TAVI) procedure on a native aortic valve. The valve-delivery-assist tool (20, 120, 220, 320) includes a delivery catheter (22); an aortic-valve cusp guide (24), which deflects laterally upon being released from the delivery catheter (22); and a conduction-tissue protector (26, 126), which includes a sheet (28, 128) and expands upon being released from a distal end (29) of the delivery catheter (22). The valve-delivery-assist tool (20, 120, 220, 320) is configured such that after lateral deflection of the aortic-valve cusp guide (24) and expansion of the conduction-tissue protector (26, 126), the released conduction-tissue protector (26, 126) and the released aortic-valve cusp guide (24) are axially stationary with respect to each other such that a distal-most point (30) of the released conduction-tissue protector (26, 126) is disposed distal to a distal-most point (32) of the released aortic-valve cusp guide (24). Other embodiments are also described.

### CROSS-REFERENCE TO RELATED APPLICATIONS

The present patent application claims priority from U.S. Provisional Application 63/070,300, filed Aug. 26, 2020, which is incorporated herein by reference.

### FIELD OF THE APPLICATION

The present invention relates generally to medical tools, and specifically to transcatheter aortic valve implantation accessories.

### BACKGROUND OF THE APPLICATION

The current standard of care for aortic valve stenosis in high-risk patients is transcatheter aortic valve implantation (TAVI), which includes the implantation of a self-expandable or balloon-expandable valve prosthesis. During TAVI procedures the valve prosthesis can interfere with the tissue surrounding the aortic valve apparatus. The manipulation and interaction of the valve prosthesis frame with conduction tissue below the aortic valve cusps in the left ventricular outflow tract (LVOT) during valve deployment can provoke heart block, including atrioventricular (AV) block (which has an incidence of up to 18% after TAVI procedures). The impingement, damage, lesion, abrasion, and/or trauma caused by the metal frame of the valve prosthesis on the conductive tissue, including the AV node, the bundle of His, and the bundle branches, can cause bradycardia that requires temporary or permanent pacemaker implantation. During TAVI procedures the height of valve implantation is a determinant of risk of paravalvular leakage (PVL). Precise implantation depth generally reduces the occurrence of PVL.

### SUMMARY OF THE APPLICATION

Some embodiments of the present invention provide a valve-delivery-assist tool for use during a transcatheter aortic valve implantation (TAVI) procedure on a native aortic valve. The valve-delivery-assist tool comprises a delivery catheter, an aortic-valve cusp guide, and a conduction-tissue protector comprising a sheet. The aortic-valve cusp guide is releasably disposed in the delivery catheter, and the valve-delivery-assist tool is configured such that the aortic-valve cusp guide deflects laterally upon being released from the delivery catheter. The conduction-tissue protector is releasably disposed compressed in the delivery catheter, and is configured to expand upon being released from a distal end of the delivery catheter.

The valve-delivery-assist tool is typically configured such that after lateral deflection of the aortic-valve cusp guide and expansion of the conduction-tissue protector, the released conduction-tissue protector and the released aortic-valve cusp guide are axially stationary with respect to each other such that a distal-most point of the released conduction-tissue protector is disposed distal to a distal-most point of the released aortic-valve cusp guide.

The valve-delivery-assist tool is typically configured such that disposition of the released aortic-valve cusp guide into a non-coronary cusp of the native aortic valve and rotational centering of the aortic-valve cusp guide within the non-coronary cusp, axially and rotationally align the released conduction-tissue protector to face a septal wall of a left ventricular outflow tract (LVOT). The conduction-tissue protector temporarily protects, by mechanically shielding, conduction tissue of the AV node from mechanical compression of the LVOT by a prosthetic aortic valve during the TAVI procedure, and/or from impingement, damage, lesion, abrasion, and/or trauma caused by the metal frame of the prosthetic aortic valve on the conductive tissue.

For some applications, the valve-delivery-assist tool further comprises a landing-support stabilizer, which is releasably disposed compressed in the delivery catheter and configured to expand (e.g., deflect laterally) after being released from the distal end of the delivery catheter. Typically, the valve-delivery-assist tool is configured such that disposition of the released aortic-valve cusp guide within a non-coronary cusp (NCC) of the native aortic valve and rotationally centering the aortic-valve cusp guide within the NCC, axially disposes the released landing-support stabilizer at a predetermined height below the native aortic valve. The predetermined height serves to define the depth of the subsequent TAVI implantation of the prosthetic aortic valve. This depth limiting may reduce the risk of paravalvular leakage (PVL) that may be caused by inappropriate depth of implantation.

For some applications, the valve-delivery-assist tool further comprises a safety tip for assisting with crossing the native valve and providing ventricular access.

There is therefore provided, in accordance with an Inventive Concept 1 of the present invention, a valve-delivery-assist tool for use during a transcatheter aortic valve implantation (TAVI) procedure on a native aortic valve, the valve-delivery-assist tool including:
a delivery catheter;
an aortic-valve cusp guide, which is releasably disposed in the delivery catheter, wherein the valve-delivery-assist tool is configured such that the aortic-valve cusp guide deflects laterally upon being released from the delivery catheter; and
a conduction-tissue protector, which (a) includes a sheet, (b) is releasably disposed compressed in the delivery catheter, and (c) is configured to expand upon being released from a distal end of the delivery catheter,
wherein the valve-delivery-assist tool is configured such that after lateral deflection of the aortic-valve cusp guide and expansion of the conduction-tissue protector, the released conduction-tissue protector and the released aortic-valve cusp guide are axially stationary with respect to each other such that a distal-most point of the released conduction-tissue protector is disposed distal to a distal-most point of the released aortic-valve cusp guide.

Inventive Concept 2. The valve-delivery-assist tool according to Inventive Concept 1, wherein the valve-delivery-assist tool is configured such that disposition of the released aortic-valve cusp guide into a non-coronary cusp of the native aortic valve and rotational centering of the aortic-valve cusp guide within the non-coronary cusp, axially and rotationally align the released conduction-tissue protector to face a septal wall of a left ventricular outflow tract (LVOT).

Inventive Concept 3. The valve-delivery-assist tool according to Inventive Concept 1, wherein the valve-delivery-assist tool is configured such that after lateral deflection of the aortic-valve cusp guide and expansion of the conduction-tissue protector, the released conduction-tissue protector and the released aortic-valve cusp guide are axially stationary with respect to each other such that the distal-most point of the released conduction-tissue protector is disposed between 2 and 30 mm distal to the distal-most point of the released aortic-valve cusp guide.

Inventive Concept 4. The valve-delivery-assist tool according to Inventive Concept 1, wherein the valve-delivery-assist tool is configured such that after lateral deflection of the aortic-valve cusp guide and expansion of the conduction-tissue protector, the released conduction-tissue protector and the released aortic-valve cusp guide are axially stationary with respect to each other such that a proximal-most point of the released conduction-tissue protector is disposed distal to, or no more than 5 mm proximal to, the distal-most point of the released aortic-valve cusp guide.

Inventive Concept 5. The valve-delivery-assist tool according to Inventive Concept 1, wherein the valve-delivery-assist tool is configured such that after lateral deflection of the aortic-valve cusp guide and expansion of the conduction-tissue protector, the released conduction-tissue protector and the released aortic-valve cusp guide are rotationally stationary with respect to each other.

Inventive Concept 6. The valve-delivery-assist tool according to Inventive Concept 1, wherein the conduction-tissue protector further includes a frame supporting the sheet.

Inventive Concept 7. The valve-delivery-assist tool according to Inventive Concept 6, wherein the frame surrounds the sheet of the released conduction-tissue protector.

Inventive Concept 8. The valve-delivery-assist tool according to Inventive Concept 6, wherein the frame includes one or more wires.

Inventive Concept 9. The valve-delivery-assist tool according to Inventive Concept 1, wherein the released conduction-tissue protector is configured such that the sheet is generally planar.

Inventive Concept 10. The valve-delivery-assist tool according to Inventive Concept 1, wherein the sheet includes a mesh.

Inventive Concept 11. The valve-delivery-assist tool according to Inventive Concept 10, wherein the mesh includes braided wires.

Inventive Concept 12. The valve-delivery-assist tool according to any one of Inventive Concepts 1-11, wherein the aortic-valve cusp guide includes an elongate slender rod.

Inventive Concept 13. The valve-delivery-assist tool according to Inventive Concept 12, wherein the elongate slender rod of the released aortic-valve cusp guide is configured to be shaped as a pigtail.

Inventive Concept 14. The valve-delivery-assist tool according to any one of Inventive Concepts 1-11, wherein the valve-delivery-assist tool is configured such that a best-fit plane defined by the released aortic-valve cusp guide forms an angle of between 15 and 60 degrees with a best-fit plane defined by the released conduction-tissue protector.

Inventive Concept 15. The valve-delivery-assist tool according to Inventive Concept 14, wherein the valve-delivery-assist tool is configured such that the angle is between 25 and 35 degrees.

Inventive Concept 16. The valve-delivery-assist tool according to any one of Inventive Concepts 1-11, further including an elongate valve-delivery-assist support, which links together the conduction-tissue protector and the aortic-valve cusp guide.

Inventive Concept 17. The valve-delivery-assist tool according to Inventive Concept 16, wherein the released aortic-valve cusp guide extends laterally from the elongate valve-10 delivery-assist support.

Inventive Concept 18. The valve-delivery-assist tool according to Inventive Concept 16, wherein the valve-delivery-assist tool is configured such that the released conduction-tissue protector extends from the elongate valve-delivery-assist support in two generally opposite directions.

Inventive Concept 19. The valve-delivery-assist tool according to Inventive Concept 16, wherein the valve-delivery-assist tool is configured such that an entirety of the released conduction-tissue protector is rotationally fixed with respect to the elongate valve-delivery-assist support.

Inventive Concept 20. The valve-delivery-assist tool according to Inventive Concept 16, wherein the elongate valve-delivery-assist support includes:
an elongate conduction-tissue-protector shaft, which is fixed to and supports the released conduction-tissue protector; and
an elongate aortic-valve cusp-guide shaft, which is fixed to and supports the released aortic-valve cusp guide,
wherein the elongate conduction-tissue-protector shaft and the elongate aortic-valve cusp-guide shaft are fixed together at least partially alongside each other.

Inventive Concept 21. The valve-delivery-assist tool according to any one of Inventive Concepts 1-11, further including a landing-support stabilizer, which is releasably disposed compressed in the delivery catheter and configured to expand after being released from the distal end of the delivery catheter,
wherein the valve-delivery-assist tool is configured such that after lateral deflection of the aortic-valve cusp guide and expansion of the conduction-tissue protector and the landing-support stabilizer:
the released landing-support stabilizer, the released conduction-tissue protector, and the released aortic-valve cusp guide are axially stationary with respect to one another such that a proximal-most point of the released landing-support stabilizer is disposed (a) distal to the distal-most point of the released aortic-valve cusp guide and (b) proximal to the distal-most point of the released conduction-tissue protector.

Inventive Concept 22. The valve-delivery-assist tool according to Inventive Concept 21, wherein the landing-support stabilizer is configured to automatically expand upon being released from the distal end of the delivery catheter.

Inventive Concept 23. The valve-delivery-assist tool according to Inventive Concept 21, wherein the landing-support stabilizer is configured to be manually expanded after being released from the distal end of the delivery catheter.

Inventive Concept 24. The valve-delivery-assist tool according to Inventive Concept 21, wherein the valve-delivery-assist tool is configured such that after lateral deflection of the aortic-valve cusp guide and expansion of the conduction-tissue protector and the landing-support stabilizer, the released landing-support stabilizer, the released conduction-tissue protector, and the released aortic-valve cusp guide are axially stationary with respect to one another such that the proximal-most point of the released landing-support stabilizer is disposed between 1 and 10 mm distal to the distal-most point of the released aortic-valve cusp guide.

Inventive Concept 25. The valve-delivery-assist tool according to Inventive Concept 24, wherein the valve-delivery-assist tool is configured such that after lateral deflection of the aortic-valve cusp guide and expansion of the conduction-tissue protector and the landing-support stabilizer, the released landing-support stabilizer, the released conduction-tissue protector, and the released aortic-valve cusp guide are axially stationary with respect to one another such that the proximal-most point of the released landing-support stabilizer is disposed between 1.5 and 4 mm distal to the distal-most point of the released aortic-valve cusp guide.

Inventive Concept 26. The valve-delivery-assist tool according to Inventive Concept 21, wherein the valve-delivery-assist tool is configured such that disposition of the released aortic-valve cusp guide within a non-coronary cusp of the native aortic valve and rotationally centering the aortic-valve cusp guide within the non-coronary cusp, axially disposes the released landing-support stabilizer at a predetermined height below the native aortic valve.

Inventive Concept 27. The valve-delivery-assist tool according to Inventive Concept 26, wherein the predetermined height is between 2 and 10 mm.

Inventive Concept 28. The valve-delivery-assist tool according to Inventive Concept 21, wherein the valve-delivery-assist tool is configured such that after lateral deflection of the aortic-valve cusp guide and expansion of the conduction-tissue protector and the landing-support stabilizer:
the released conduction-tissue protector and the released aortic-valve cusp guide are rotationally stationary with respect to one another.

Inventive Concept 29. The valve-delivery-assist tool according to Inventive Concept 28, wherein the valve-delivery-assist tool is configured such that after lateral deflection of the aortic-valve cusp guide and expansion of the conduction-tissue protector and the landing-support stabilizer:
the released landing-support stabilizer, the released conduction-tissue protector, and the released aortic-valve cusp guide are rotationally stationary with respect to one another.

Inventive Concept 30. The valve-delivery-assist tool according to Inventive Concept 21, wherein the valve-delivery-assist tool is configured such that after lateral deflection of the aortic-valve cusp guide and expansion of the conduction-tissue protector and the landing-support stabilizer:
the released landing-support stabilizer and the released conduction-tissue protector are rotationally stationary with respect to one another.

Inventive Concept 31. The valve-delivery-assist tool according to Inventive Concept 21, wherein the valve-delivery-assist tool is configured such that the released landing-support stabilizer and the released aortic-valve cusp guide are disposed on opposite sides of the released conduction-tissue protector.

Inventive Concept 32. The valve-delivery-assist tool according to Inventive Concept 21, wherein the landing-support stabilizer includes an elongate slender rod.

Inventive Concept 33. The valve-delivery-assist tool according to Inventive Concept 32, wherein the elongate slender rod of the released landing-support stabilizer is shaped as a partial loop or a complete loop having one or more turns.

Inventive Concept 34. The valve-delivery-assist tool according to Inventive Concept 33, wherein the valve-delivery-assist tool further includes (a) an elongate landing-support-stabilizer shaft, which is fixed to and supports the landing-support stabilizer, and (b) an elongate support shaft, and
wherein a distal end of the elongate slender rod is fixed to the elongate support shaft such that:
   disposal of the elongate support shaft in a more distal position stretches the elongate slender rod and radially compresses the elongate slender rod against the elongate support shaft, and
   disposal of the elongate support shaft in a more proximal position causes the elongate slender rod to bulge away from, and radially expand with respect to, the elongate support shaft.

Inventive Concept 35. The valve-delivery-assist tool according to Inventive Concept 33, wherein the valve-delivery-assist tool further includes (a) an elongate landing-support-stabilizer shaft, which is fixed to and supports the landing-support stabilizer, and (b) an elongate support shaft, which is shaped so as to define a channel having a distal opening through which the elongate slender rod is slidable, and
wherein the valve-delivery-assist tool is configured such that:
   withdrawal of the elongate slender rod into the channel radially compresses the elongate slender rod within the elongate support shaft, and
   extension of the elongate slender rod from the channel causes the elongate slender rod to bulge away from, and radially expand with respect to, the elongate support shaft.

Inventive Concept 36. The valve-delivery-assist tool according to Inventive Concept 21, further including an elongate valve-delivery-assist support, which links together the landing-support stabilizer, the conduction-tissue protector, and the aortic-valve cusp guide.

Inventive Concept 37. The valve-delivery-assist tool according to Inventive Concept 36, wherein the released landing-support stabilizer extends laterally from the elongate valve-delivery-assist support.

Inventive Concept 38. The valve-delivery-assist tool according to Inventive Concept 37, wherein a best-fit plane defined by the released landing-support stabilizer forms an angle of between 45 and 90 degrees with a central longitudinal axis of the elongate valve-delivery-assist support that is straight and passes through the distal end of the elongate valve-delivery-assist support.

Inventive Concept 39. The valve-delivery-assist tool according to Inventive Concept 36, wherein the elongate valve-delivery-assist support includes:
an elongate conduction-tissue-protector shaft, which is fixed to and supports the conduction-tissue protector;
an elongate aortic-valve cusp-guide shaft, which is fixed to and supports the aortic-valve cusp guide; and
an elongate landing-support-stabilizer shaft, which is fixed to and supports the landing-support stabilizer,
wherein the elongate conduction-tissue-protector shaft, the elongate aortic-valve cusp-guide shaft, and the elongate landing-support-stabilizer shaft are fixed together at least partially alongside one another.

Inventive Concept 40. The valve-delivery-assist tool according to Inventive Concept 21, wherein the valve-delivery-assist tool is configured such that a best-fit plane defined by the released landing-support stabilizer forms an angle of between 45 and 90 degrees with a best-fit plane defined by the released conduction-tissue protector.

Inventive Concept 41. The valve-delivery-assist tool according to Inventive Concept 40, wherein the valve-delivery-assist tool is configured such that a best-fit plane defined by the released aortic-valve cusp guide forms an angle of between 45 and 90 degrees with the best-fit plane defined by the released landing-support stabilizer.

Inventive Concept 42. The valve-delivery-assist tool according to Inventive Concept 21,
further including a safety tip, which is releasably disposed compressed in the delivery catheter, configured to be released from the distal end of the delivery catheter, and shaped so as to define a guidewire channel through the safety tip,
wherein the safety tip, the conduction-tissue protector, and the aortic-valve cusp guide are linked to one another such that after expansion of the conduction-tissue protector, a distal-most point of the safety tip is disposed distal to the distal-most point of the released conduction-tissue protector.

Inventive Concept 43. The valve-delivery-assist tool according to Inventive Concept 42, wherein the safety tip is configured to expand upon released from the distal end of the delivery catheter.

Inventive Concept 44. The valve-delivery-assist tool according to Inventive Concept 42, wherein the safety tip includes an elongate slender rod.

Inventive Concept 45. The valve-delivery-assist tool according to Inventive Concept 44, wherein the elongate slender rod of the released safety tip is configured to be shaped as a pigtail.

Inventive Concept 46. The valve-delivery-assist tool according to Inventive Concept 42, wherein the safety tip and the conduction-tissue protector are integral to each other.

Inventive Concept 47. The valve-delivery-assist tool according to any one of Inventive Concepts 1-11, for use with a guidewire,
wherein the valve-delivery-assist tool further includes a nose cone, which is shaped so as to define (a) a wider proximal base that is removably coupled to a distal end of the delivery catheter, and (b) a narrower elongate distal safety tip that extends distally from the proximal base,
wherein the nose cone is shaped so as to define a guidewire channel through the proximal base and the elongate distal safety tip, and
wherein the elongate distal safety tip is configured to assume a straight shape when the guidewire is disposed in the guidewire channel, and a curved shape when the guidewire is not disposed in the guidewire channel.

Inventive Concept 48. The valve-delivery-assist tool according to Inventive Concept 47, wherein the curved shape is a pigtail shape.

Inventive Concept 49. The valve-delivery-assist tool according to any one of Inventive Concepts 1-11,
further including a safety tip, which is releasably disposed compressed in the delivery catheter, configured to be released from the distal end of the delivery catheter, and shaped so as to define a guidewire channel through the safety tip,
wherein the safety tip, the conduction-tissue protector, and the aortic-valve cusp guide are linked to one another such that after expansion of the conduction-tissue protector, a distal-most point of the safety tip is disposed distal to the distal-most point of the released conduction-tissue protector.

Inventive Concept 50. The valve-delivery-assist tool according to Inventive Concept 49, wherein the safety tip is configured to expand upon released from the distal end of the delivery catheter.

Inventive Concept 51. The valve-delivery-assist tool according to Inventive Concept 49, wherein the safety tip includes an elongate slender rod.

Inventive Concept 52. The valve-delivery-assist tool according to Inventive Concept 51, wherein the elongate slender rod of the released safety tip is configured to be shaped as a pigtail.

Inventive Concept 53. The valve-delivery-assist tool according to Inventive Concept 49, wherein the safety tip and the conduction-tissue protector are integral to each other.

Inventive Concept 54. The valve-delivery-assist tool according to any one of Inventive Concepts 1-11, further including a reshaping element, which is coupled to the conduction-15 tissue protector, which is configured to change a shape of the released conduction-tissue protector.

Inventive Concept 55. The valve-delivery-assist tool according to Inventive Concept 54,
wherein the conduction-tissue protector further includes a frame supporting the sheet, and
wherein the reshaping element is coupled to the frame of the conduction-tissue protector.

Inventive Concept 56. The valve-delivery-assist tool according to Inventive Concept 54, wherein the reshaping element includes an elongate reshaping shaft.

Inventive Concept 57. The valve-delivery-assist tool according to any one of Inventive Concepts 1-11, wherein the valve-delivery-assist tool is shaped so to define a contrast channel, which is shaped so as to define one or more lateral or distal openings for injection of contrast media.

Inventive Concept 58. The valve-delivery-assist tool according to Inventive Concept 57, wherein the delivery catheter is shaped so as to define the contrast channel.

Inventive Concept 59. The valve-delivery-assist tool according to Inventive Concept 57, wherein the valve-delivery-assist tool includes an elongate shaft that is shaped so as to define the contrast channel.

Inventive Concept 60. The valve-delivery-assist tool according to any one of Inventive Concepts 1-11, wherein the delivery catheter includes an embolic protector structure, which includes a filter or mesh, and which can be elongated and opened proximally to a distal end of the embolic protector structure.

There is further provided, in accordance with an Inventive Concept 61 of the present invention, a valve-delivery-assist tool for use during a transcatheter aortic valve implantation (TAVI) procedure on a native aortic valve, the valve-delivery-assist tool including:
a delivery catheter;
an aortic-valve cusp guide, which is releasably disposed in the delivery catheter, wherein the valve-delivery-assist tool is configured such that the aortic-valve cusp guide deflects laterally upon being released from the delivery catheter; and
a landing-support stabilizer, which is releasably disposed compressed in the delivery catheter and configured to expand after being released from a distal end of the delivery catheter.

Inventive Concept 62. The valve-delivery-assist tool according to Inventive Concept 61, wherein the landing-support stabilizer is configured to automatically expand upon being released from the distal end of the delivery catheter.

Inventive Concept 63. The valve-delivery-assist tool according to Inventive Concept 61, wherein the landing-support stabilizer is configured to be manually expanded after being released from the distal end of the delivery catheter.

Inventive Concept 64. The valve-delivery-assist tool according to Inventive Concept 61, wherein the valve-delivery-assist tool is configured such that after lateral deflection of the aortic-valve cusp guide and expansion of the landing-support stabilizer, the released landing-support stabilizer and the released aortic-valve cusp guide are axially stationary with respect to one another such that a proximal-most point of the released landing-support stabilizer is disposed distal to the distal-most point of the released aortic-valve cusp guide.

Inventive Concept 65. The valve-delivery-assist tool according to Inventive Concept 64, wherein the valve-delivery-assist tool is configured such that after lateral deflection of the aortic-valve cusp guide and expansion of the landing-support stabilizer, the released landing-support stabilizer and the released aortic-valve cusp guide are axially stationary with respect to one another such that the proximal-most point of the released landing-support stabilizer is disposed between 1 and 10 mm distal to the distal-most point of the released aortic-valve cusp guide.

Inventive Concept 66. The valve-delivery-assist tool according to Inventive Concept 65, wherein the valve-delivery-assist tool is configured such that after lateral deflection of the aortic-valve cusp guide and expansion of the landing-support stabilizer, the released landing-support stabilizer and the released aortic-valve cusp guide are axially stationary with respect to one another such that the proximal-most point of the released landing-support stabilizer is disposed between 1.5 and 4 mm distal to the distal-most point of the released aortic-valve cusp guide.

Inventive Concept 67. The valve-delivery-assist tool according to Inventive Concept 61, wherein the valve-delivery-assist tool is configured such that disposition of the released aortic-valve cusp guide within a non-coronary cusp of the native aortic valve and rotationally centering the aortic-valve cusp guide within the non-coronary cusp, axially disposes the released landing-support stabilizer at a predetermined height below the native aortic valve.

Inventive Concept 68. The valve-delivery-assist tool according to Inventive Concept 67, wherein the predetermined height is between 2 and 10 mm.

Inventive Concept 69. The valve-delivery-assist tool according to Inventive Concept 61, wherein the valve-delivery-assist tool is configured such that after lateral deflection of the aortic-valve cusp guide and expansion of the landing-support stabilizer, the released landing-support stabilizer and the released aortic-valve cusp guide are rotationally stationary with respect to one another.

Inventive Concept 70. The valve-delivery-assist tool according to Inventive Concept 61, wherein the landing-support stabilizer includes an elongate slender rod.

Inventive Concept 71. The valve-delivery-assist tool according to Inventive Concept 70, wherein the elongate slender rod of the released landing-support stabilizer is shaped as a partial loop or a complete loop having one or more turns.

Inventive Concept 72. The valve-delivery-assist tool according to Inventive Concept 71,
wherein the valve-delivery-assist tool further includes (a) an elongate landing-support-stabilizer shaft, which is fixed to and supports the landing-support stabilizer, and (b) an elongate support shaft, and
wherein a distal end of the elongate slender rod is fixed to the elongate support shaft such that:
   disposal of the elongate support shaft in a more distal position stretches the elongate slender rod and radially compresses the elongate slender rod against the elongate support shaft, and
   disposal of the elongate support shaft in a more proximal position causes the elongate slender rod to bulge away from, and radially expand with respect to, the elongate support shaft.

Inventive Concept 73. The valve-delivery-assist tool according to Inventive Concept 71,
wherein the valve-delivery-assist tool further includes (a) an elongate landing-support-stabilizer shaft, which is fixed to and supports the landing-support stabilizer, and (b) an elongate support shaft, which is shaped so as to define a channel having a distal opening through which the elongate slender rod is slidable, and
wherein the valve-delivery-assist tool is configured such that:
   withdrawal of the elongate slender rod into the channel radially compresses the elongate slender rod within the elongate support shaft, and
   extension of the elongate slender rod from the channel causes the elongate slender rod to bulge away from, and radially expand with respect to, the elongate support shaft.

Inventive Concept 74. The valve-delivery-assist tool according to Inventive Concept 61, further including an elongate valve-delivery-assist support, which links together the landing-support stabilizer and the aortic-valve cusp guide.

Inventive Concept 75. The valve-delivery-assist tool according to Inventive Concept 74, wherein the released landing-support stabilizer extends laterally from the elongate valve-delivery-assist support.

Inventive Concept 76. The valve-delivery-assist tool according to Inventive Concept 75, wherein a best-fit plane defined by the released landing-support stabilizer forms an angle of between 45 and 90 degrees with a central longitudinal axis of the elongate valve-delivery assist support that is straight and passes through the distal end of the elongate valve-delivery-assist support.

Inventive Concept 77. The valve-delivery-assist tool according to Inventive Concept 74, wherein the elongate valve-delivery-assist support includes:
an elongate aortic-valve cusp-guide shaft, which is fixed to and supports the aortic-valve cusp guide; and
an elongate landing-support-stabilizer shaft, which is fixed to and supports the landing-support stabilizer,
wherein the elongate aortic-valve cusp-guide shaft and the elongate landing-support-stabilizer shaft are fixed together at least partially alongside one another.

Inventive Concept 78. The valve-delivery-assist tool according to any one of Inventive Concepts 61-77, for use with a guidewire,
wherein the valve-delivery-assist tool further includes a nose cone, which is shaped so as to define (a) a wider proximal base that is removably coupled to a distal end of the delivery catheter, and (b) a narrower elongate distal safety tip that extends distally from the proximal base,
wherein the nose cone is shaped so as to define a guidewire channel through the proximal base and the elongate distal safety tip, and
wherein the elongate distal safety tip is configured to assume a straight shape when the guidewire is disposed in the guidewire channel, and a curved shape when the guidewire is not disposed in the guidewire channel.

Inventive Concept 79. The valve-delivery-assist tool according to Inventive Concept 78, wherein the curved shape is a pigtail shape.

Inventive Concept 80. The valve-delivery-assist tool according to any one of Inventive Concepts 61-77, further including a safety tip, which is releasably disposed compressed in the delivery catheter, configured to be released from the distal end of the delivery catheter, and shaped so as to define a guidewire channel through the safety tip.

Inventive Concept 81. The valve-delivery-assist tool according to Inventive Concept 80, wherein the safety tip is configured to expand upon released from the distal end of the delivery catheter.

Inventive Concept 82. The valve-delivery-assist tool according to Inventive Concept 80, wherein the safety tip includes an elongate slender rod.

Inventive Concept 83. The valve-delivery-assist tool according to Inventive Concept 82, wherein the elongate slender rod of the released safety tip is configured to be shaped as a pigtail.

There is still further provided, in accordance with an Inventive Concept 84 of the present invention, a method for performing a transcatheter aortic valve implantation (TAVI) procedure on a native aortic valve, the method including:
advancing a delivery catheter of a valve-delivery-assist tool into an aorta and partially into a left ventricle, while an aortic-valve cusp guide and a conduction-tissue protector of the valve-delivery-assist tool are releasably disposed in the delivery catheter;
releasing the conduction-tissue protector from a distal end of the delivery catheter, such that the conduction-tissue protector expands to a deployed shape in the left ventricle, the conduction-tissue protector including a sheet;
releasing the aortic-valve cusp guide from the distal end of the delivery catheter, such that (a) the aortic-valve cusp guide deflects laterally and transitions to a deployed shape in an ascending aorta and is seated within one of aortic valve cusps of the native aortic valve, and (b) the released conduction-tissue protector and the released aortic-valve cusp guide are axially stationary with respect to each other such that (i) a distal-most point of the released conduction-tissue protector is disposed distal to a distal-most point of the released aortic-valve cusp guide, and (ii) rotational centering of the aortic-valve cusp guide within the aortic valve cusp axially and rotationally aligns the released conduction-tissue protector to face a septal wall of a left ventricular outflow tract;
thereafter, deploying a prosthetic aortic valve, while the deployed conduction-tissue protector protects, by mechanically shielding, conduction tissue of an AV node from mechanical compression by the prosthetic aortic valve; and
thereafter, withdrawing the valve-delivery-assist tool, leaving the prosthetic aortic valve implanted in the native aortic valve.

Inventive Concept 85. The method according to Inventive Concept 84, wherein advancing the delivery catheter partially into the left ventricle includes seating the delivery catheter in a commissure between two of the aortic valve cusps.

Inventive Concept 86. The method according to Inventive Concept 84, wherein the one of the aortic valve cusps is a non-coronary cusp.

Inventive Concept 87. The method according to Inventive Concept 84, wherein the valve-delivery-assist tool is configured such that after lateral deflection of the aortic-valve cusp guide and expansion of the conduction-tissue protector, the released conduction-tissue protector and the released aortic-valve cusp guide are rotationally stationary with respect to each other.

Inventive Concept 88. The method according to Inventive Concept 84, wherein the conduction-tissue protector further includes a frame supporting the sheet.

Inventive Concept 89. The method according to Inventive Concept 88, wherein the frame surrounds the sheet of the released conduction-tissue protector.

Inventive Concept 90. The method according to Inventive Concept 84, wherein releasing the conduction-tissue protector includes releasing the conduction-tissue protector includes such that the sheet is generally planar.

Inventive Concept 91. The method according to Inventive Concept 84, wherein the sheet includes a mesh.

Inventive Concept 92. The method according to Inventive Concept 91, wherein the mesh includes braided wires.

Inventive Concept 93. The method according to Inventive Concept 84, wherein the aortic-valve cusp guide includes an elongate slender rod.

Inventive Concept 94. The method according to Inventive Concept 93, wherein the elongate slender rod of the released aortic-valve cusp guide is configured to be shaped as a pigtail.

Inventive Concept 95. The method according to Inventive Concept 84, wherein releasing the aortic-valve cusp guide includes releasing the aortic-valve cusp guide such that a best-fit plane defined by the released aortic-valve cusp guide forms an angle of between 15 and 60 degrees with a best-fit plane defined by the released conduction-tissue protector.

Inventive Concept 96. The method according to Inventive Concept 95, wherein the angle is between 25 and 35 degrees.

Inventive Concept 97. The method according to Inventive Concept 84, wherein the valve-delivery-assist tool further includes an elongate valve-delivery-assist support, which links together the conduction-tissue protector and the aortic-valve cusp guide.

Inventive Concept 98. The method according to Inventive Concept 97, wherein the released aortic-valve cusp guide extends laterally from the elongate valve-delivery-assist support.

Inventive Concept 99. The method according to Inventive Concept 97, wherein releasing the released conduction-tissue protector includes releasing the released conduction-tissue protector such that the released conduction-tissue protector extends from the elongate valve-delivery-assist support in two generally opposite directions.

Inventive Concept 100. The method according to Inventive Concept 97, wherein the valve-delivery-assist tool is configured such that an entirety of the released conduction-10 tissue protector is rotationally fixed with respect to the elongate valve-delivery-assist support.

Inventive Concept 101. The method according to Inventive Concept 97, wherein the elongate valve-delivery-assist support includes:
an elongate conduction-tissue-protector shaft, which is fixed to and supports the released conduction-tissue protector; and
an elongate aortic-valve cusp-guide shaft, which is fixed to and supports the released aortic-valve cusp guide,
wherein the elongate conduction-tissue-protector shaft and the elongate aortic-valve cusp-guide shaft are fixed together at least partially alongside each other.

Inventive Concept 102. The method according to Inventive Concept 84,
wherein advancing the delivery catheter includes advancing the delivery catheter while the aortic-valve cusp guide, the conduction-tissue protector, and a landing-support stabilizer of the valve-delivery-assist tool are releasably disposed in the delivery catheter, and
wherein the method further includes releasing the landing-support stabilizer from the distal end of the delivery catheter in the left ventricle, after which the landing-support stabilizer expands and the released landing-support stabilizer, the released conduction-tissue protector, and the released aortic-valve cusp guide are axially stationary with respect to one another such that:
   a proximal-most point of the released landing-support stabilizer is disposed (a) distal to the distal-most point of the released aortic-valve cusp guide and (b) proximal to the distal-most point of the released conduction-tissue protector,
   the landing-support stabilizer is axially disposed at a predetermined height below native aortic valve, and
wherein deploying the prosthetic aortic valve includes deploying the prosthetic aortic valve while the landing-support stabilizer limits a depth of implantation of the prosthetic aortic valve to the predetermined height below the native aortic valve.

Inventive Concept 103. The method according to Inventive Concept 102, wherein the predetermined height is between 2 and 10 mm.

Inventive Concept 104. The method according to Inventive Concept 102, wherein the landing-support stabilizer is configured to automatically expand upon being released from the distal end of the delivery catheter.

Inventive Concept 105. The method according to Inventive Concept 102, wherein releasing the landing-support stabilizer from the distal end of the delivery catheter in the left ventricle includes manually expanding the landing-support stabilizer.

Inventive Concept 106. The method according to Inventive Concept 102, wherein the valve-delivery-assist tool is configured such that after lateral deflection of the aortic-valve cusp guide and expansion of the conduction-tissue protector and the landing-support stabilizer:
the released conduction-tissue protector and the released aortic-valve cusp guide are rotationally stationary with respect to one another.

Inventive Concept 107. The method according to Inventive Concept 106, wherein the valve-delivery-assist tool is configured such that after lateral deflection of the aortic-valve cusp guide and expansion of the conduction-tissue protector and the landing-support stabilizer:
the released landing-support stabilizer, the released conduction-tissue protector, and the released aortic-valve cusp guide are rotationally stationary with respect to one another.

Inventive Concept 108. The method according to Inventive Concept 102, wherein the valve-delivery-assist tool is configured such that after lateral deflection of the aortic-valve cusp guide and expansion of the conduction-tissue protector and the landing-support stabilizer:
the released landing-support stabilizer and the released conduction-tissue protector are rotationally stationary with respect to one another.

Inventive Concept 109. The method according to Inventive Concept 102, wherein the valve-delivery-assist tool is configured such that the released landing-support stabilizer and the released aortic-valve cusp guide are disposed on opposite sides of the released conduction-tissue protector.

Inventive Concept 110. The method according to Inventive Concept 102, wherein the landing-support stabilizer includes an elongate slender rod.

Inventive Concept 111. The method according to Inventive Concept 110, wherein the elongate slender rod of the released landing-support stabilizer is shaped as a partial loop or a complete loop having one or more turns.

Inventive Concept 112. The method according to Inventive Concept 111,
wherein the valve-delivery-assist tool further includes (a) an elongate landing-support-stabilizer shaft, which is fixed to and supports the landing-support stabilizer, and (b) an elongate support shaft,
wherein a distal end of the elongate slender rod is fixed to the elongate support shaft,
wherein advancing the delivery catheter includes advancing the delivery catheter while the elongate support shaft is disposed in a more distal position, stretching the elongate slender rod and radially compresses the elongate slender rod against the elongate support shaft, and
wherein releasing the landing-support stabilizer from the distal end of the delivery catheter in the left ventricle includes disposing the elongate support shaft in a more proximal position so as to cause the elongate slender rod to bulge away from, and radially expand with respect to, the elongate support shaft.

Inventive Concept 113. The method according to Inventive Concept 111,
wherein the valve-delivery-assist tool further includes (a) an elongate landing-support-stabilizer shaft, which is fixed to and supports the landing-support stabilizer, and (b) an elongate support shaft, which is shaped so as to define a channel having a distal opening through which the elongate slender rod is slidable,
wherein advancing the delivery catheter includes advancing the delivery catheter while the elongate slender rod is withdrawn into the channel, thereby radially compressing the elongate slender rod within the elongate support shaft, and
wherein releasing the landing-support stabilizer from the distal end of the delivery catheter in the left ventricle includes extending the elongate slender rod from the channel so as to cause the elongate slender rod to bulge away from, and radially expand with respect to, the elongate support shaft.

Inventive Concept 114. The method according to Inventive Concept 102, wherein the valve-delivery-assist tool further includes an elongate valve-delivery-assist support, which links together the landing-support stabilizer, the conduction-tissue protector, and the aortic-valve cusp guide.

Inventive Concept 115. The method according to Inventive Concept 114, wherein the released landing-support stabilizer extends laterally from the elongate valve-delivery-assist support.

Inventive Concept 116. The method according to Inventive Concept 115, wherein releasing the landing-support stabilizer includes releasing the landing-support stabilizer such that a best-fit plane defined by the released landing-support stabilizer forms an angle of between 45 and 90 degrees with a central longitudinal axis of the elongate valve-delivery-assist support that is straight and passes through the distal end of the elongate valve-delivery-assist support.

Inventive Concept 117. The method according to Inventive Concept 114, wherein the elongate valve-delivery-assist support includes:
an elongate conduction-tissue-protector shaft, which is fixed to and supports the conduction-tissue protector;
an elongate aortic-valve cusp-guide shaft, which is fixed to and supports the aortic-valve cusp guide; and
an elongate landing-support-stabilizer shaft, which is fixed to and supports the landing-support stabilizer,
wherein the elongate conduction-tissue-protector shaft, the elongate aortic-valve cusp-guide shaft, and the elongate landing-support-stabilizer shaft are fixed together at least partially alongside one another.

Inventive Concept 118. The method according to Inventive Concept 102, wherein releasing the landing-support stabilizer includes releasing the landing-support stabilizer such that a best-fit plane defined by the released landing-support stabilizer forms an angle of between 45 and 90 degrees with a best-fit plane defined by the released conduction-tissue protector.

Inventive Concept 119. The method according to Inventive Concept 118, wherein releasing the aortic-valve cusp guide includes releasing the aortic-valve cusp guide such that a best-fit plane defined by the released aortic-valve cusp guide forms an angle of between 45 and 90 degrees with the best-fit plane defined by the released landing-support stabilizer.

Inventive Concept 120. The method according to Inventive Concept 84,
wherein advancing the delivery catheter includes advancing the delivery catheter while the aortic-valve cusp guide, the conduction-tissue protector, and a safety tip of the valve-delivery-assist tool are releasably disposed in the delivery catheter, the safety tip shaped so as to define a guidewire channel through the safety tip,
wherein the method further includes releasing the safety tip from the distal end of the delivery catheter in the left ventricle such that the safety tip transitions to a deployed shape in the left ventricle, and
wherein the safety tip, the conduction-tissue protector, and the aortic-valve cusp guide are linked to one another such that after expansion of the conduction-tissue protector, a distal-most point of the safety tip is disposed distal to the distal-most point of the released conduction-tissue protector.

Inventive Concept 121. The method according to Inventive Concept 120, wherein releasing the safety tip includes releasing the safety tip such that it expands.

Inventive Concept 122. The method according to Inventive Concept 120, wherein the safety tip includes an elongate slender rod.

Inventive Concept 123. The method according to Inventive Concept 122, wherein the elongate slender rod of the released safety tip is configured to be shaped as a pigtail.

Inventive Concept 124. The method according to Inventive Concept 120, wherein the safety tip and the conduction-tissue protector are integral to each other.

Inventive Concept 125. The method according to Inventive Concept 84, wherein advancing the delivery catheter includes advancing a guidewire between the aortic valve cusps into the left ventricle, and advancing the delivery catheter over the guidewire.

Inventive Concept 126. The method according to Inventive Concept 84, wherein advancing the delivery catheter partially into the left ventricle includes passing a nose cone between two of the aortic valve cusps into the left ventricle while the nose cone is removably coupled to the delivery catheter.

Inventive Concept 127. The method according to Inventive Concept 126,
wherein advancing the delivery catheter includes advancing a guidewire between the aortic valve cusps into the left ventricle, and advancing the delivery catheter over the guidewire,
wherein the nose cone is shaped so as to define (a) a wider proximal base that is removably coupled to a distal end of the delivery catheter, and (b) a narrower elongate distal safety tip that extends distally from the proximal base,
wherein the nose cone is shaped so as to define a guidewire channel through the proximal base and the elongate distal safety tip,
wherein the elongate distal safety tip is configured to assume a straight shape when the guidewire is disposed in the guidewire channel, and
wherein the method further includes proximally withdrawing the guidewire from the nose cone such that the elongate distal safety tip assumes a curved shape.

Inventive Concept 128. The method according to Inventive Concept 127, wherein the curved shape is a pigtail shape.

Inventive Concept 129. The method according to Inventive Concept 84, wherein the valve-delivery-assist tool further includes a reshaping element, which is coupled to the conduction-tissue protector, which is configured to change a shape of the released conduction-tissue protector.

Inventive Concept 130. The method according to Inventive Concept 129,
wherein the conduction-tissue protector further includes a frame supporting the sheet, and
wherein the reshaping element is coupled to the frame of the conduction-tissue protector.

Inventive Concept 131. The method according to Inventive Concept 129, wherein the reshaping element includes an elongate reshaping shaft.

Inventive Concept 132. The method according to Inventive Concept 84, wherein the valve-delivery-assist tool is shaped so to define a contrast channel, which is shaped so as to define one or more lateral or distal openings, and wherein the method further includes injecting contrast media via the one or more lateral or distal openings.

Inventive Concept 133. The method according to Inventive Concept 132, wherein the delivery catheter is shaped so as to define the contrast channel.

Inventive Concept 134. The method according to Inventive Concept 132, wherein the valve-delivery-assist tool includes an elongate shaft that is shaped so as to define the contrast channel.

Inventive Concept 135. The method according to Inventive Concept 84, wherein the delivery catheter includes an embolic protector structure, which includes a filter or mesh, and wherein the method further includes elongating and opening the embolic protector structure proximally to a distal end of the embolic protector structure.

There is additionally provided, in accordance with an Inventive Concept 136 of the present invention, a method for performing a transcatheter aortic valve implantation (TAVI) procedure on a native aortic valve, the method including:
advancing a delivery catheter of a valve-delivery-assist tool into an aorta and partially into a left ventricle, while an aortic-valve cusp guide and a landing-support stabilizer of the valve-delivery-assist tool are releasably disposed in the delivery catheter;
releasing the landing-support stabilizer from a distal end of the delivery catheter, after which the landing-support stabilizer expands;
releasing the aortic-valve cusp guide from the distal end of the delivery catheter, such that (a) the aortic-valve cusp guide deflects laterally and transitions to a deployed shape in an ascending aorta and is seated within one of aortic valve cusps of the native aortic valve, and (b) the released landing-support stabilizer and the released aortic-valve cusp guide are axially stationary with respect to each other such that the landing-support stabilizer is disposed at a predetermined height below native aortic valve;
thereafter, deploying a prosthetic aortic valve while the landing-support stabilizer limits a depth of implantation of the prosthetic aortic valve to the predetermined height below the native aortic valve; and
thereafter, withdrawing the valve-delivery-assist tool, leaving the prosthetic aortic valve implanted in the native aortic valve.

Inventive Concept 137. The method according to Inventive Concept 136, wherein the landing-support stabilizer is configured to automatically expand upon being released from the distal end of the delivery catheter.

Inventive Concept 138. The method according to Inventive Concept 136, wherein releasing the landing-support stabilizer from the distal end of the delivery catheter in the left ventricle includes manually expanding the landing-support stabilizer.

Inventive Concept 139. The method according to Inventive Concept 136, wherein advancing the delivery catheter partially into the left ventricle includes passing a nose cone between two of the aortic valve cusps into the left ventricle while the nose cone is removably coupled to the delivery catheter.

Inventive Concept 140. The method according to Inventive Concept 136, wherein advancing the delivery catheter partially into the left ventricle includes seating the delivery catheter in a commissure between two of the aortic valve cusps.

Inventive Concept 141. The method according to Inventive Concept 136, wherein the one of the aortic valve cusps is a non-coronary cusp.

Inventive Concept 142. The method according to Inventive Concept 136, wherein releasing the landing-support stabilizer and the aortic-valve cusp guide includes releasing the landing-support stabilizer and the aortic-valve cusp guide such that the released landing-support stabilizer and the released aortic-valve cusp guide are axially stationary with respect to one another such that a proximal-most point of the released landing-support stabilizer is disposed distal to the distal-most point of the released aortic-valve cusp guide.

Inventive Concept 143. The method according to Inventive Concept 136, wherein the predetermined height is between 2 and 10 mm.

Inventive Concept 144. The method according to Inventive Concept 136, wherein the valve-delivery-assist tool is configured such that after lateral deflection of the aortic-valve cusp guide and expansion of the landing-support stabilizer, the released landing-support stabilizer and the released aortic-valve cusp guide are rotationally stationary with respect to one another.

Inventive Concept 145. The method according to Inventive Concept 136, wherein the landing-support stabilizer includes an elongate slender rod.

Inventive Concept 146. The method according to Inventive Concept 145, wherein the elongate slender rod of the released landing-support stabilizer is shaped as a partial loop or a complete loop having one or more turns.

Inventive Concept 147. The method according to Inventive Concept 146,
wherein the valve-delivery-assist tool further includes (a) an elongate landing-support-stabilizer shaft, which is fixed to and supports the landing-support stabilizer, and (b) an elongate support shaft,
wherein a distal end of the elongate slender rod is fixed to the elongate support shaft,
wherein advancing the delivery catheter includes advancing the delivery catheter while the elongate support shaft is disposed in a more distal position, stretching the elongate slender rod and radially compresses the elongate slender rod against the elongate support shaft, and
wherein releasing the landing-support stabilizer from the distal end of the delivery catheter in the left ventricle includes disposing the elongate support shaft in a more proximal position so as to cause the elongate slender rod to bulge away from, and radially expand with respect to, the elongate support shaft.

Inventive Concept 148. The method according to Inventive Concept 146,
wherein the valve-delivery-assist tool further includes (a) an elongate landing-support-stabilizer shaft, which is fixed to and supports the landing-support stabilizer, and (b) an elongate support shaft, which is shaped so as to define a channel having a distal opening through which the elongate slender rod is slidable,
wherein advancing the delivery catheter includes advancing the delivery catheter while the elongate slender rod is withdrawn into the channel, thereby radially compressing the elongate slender rod within the elongate support shaft, and
wherein releasing the landing-support stabilizer from the distal end of the delivery catheter in the left ventricle includes extending the elongate slender rod from the channel so as to cause the elongate slender rod to bulge away from, and radially expand with respect to, the elongate support shaft.

Inventive Concept 149. The method according to Inventive Concept 136, wherein the valve-delivery-assist tool further includes an elongate valve-delivery-assist support, which links together the landing-support stabilizer and the aortic-valve cusp guide.

Inventive Concept 150. The method according to Inventive Concept 149, wherein the released landing-support stabilizer extends laterally from the elongate valve-delivery-assist support.

Inventive Concept 151. The method according to Inventive Concept 150, wherein releasing the landing-support stabilizer includes releasing the landing-support stabilizer such that a best-fit plane defined by the released landing-support stabilizer forms an angle of between 45 and 90 degrees with a central longitudinal axis of the elongate valve-delivery-assist support that is straight and passes through the distal end of the elongate valve-delivery-assist support.

Inventive Concept 152. The method according to Inventive Concept 149, wherein the elongate valve-delivery-assist support includes:
an elongate aortic-valve cusp-guide shaft, which is fixed to and supports the aortic-valve cusp guide; and
an elongate landing-support-stabilizer shaft, which is fixed to and supports the landing-support stabilizer,
wherein the elongate aortic-valve cusp-guide shaft and the elongate landing-support-stabilizer shaft are fixed together at least partially alongside one another.

Inventive Concept 153. The method according to Inventive Concept 136, wherein advancing the delivery catheter includes advancing a guidewire between the aortic valve cusps into the left ventricle, and advancing the delivery catheter over the guidewire.

Inventive Concept 154. The method according to Inventive Concept 136, wherein advancing the delivery catheter partially into the left ventricle includes passing a nose cone between two of the aortic valve cusps into the left ventricle while the nose cone is removably coupled to the delivery catheter.

Inventive Concept 155. The method according to Inventive Concept 154,
wherein advancing the delivery catheter includes advancing a guidewire between the aortic valve cusps into the left ventricle, and advancing the delivery catheter over the guidewire,
wherein the nose cone is shaped so as to define (a) a wider proximal base that is removably coupled to a distal end of the delivery catheter, and (b) a narrower elongate distal safety tip that extends distally from the proximal base,
wherein the nose cone is shaped so as to define a guidewire channel through the proximal base and the elongate distal safety tip,
wherein the elongate distal safety tip is configured to assume a straight shape when the guidewire is disposed in the guidewire channel, and
wherein the method further includes proximally withdrawing the guidewire from the nose cone such that the elongate distal safety tip assumes a curved shape.

Inventive Concept 156. The method according to Inventive Concept 155, wherein the curved shape is a pigtail shape.

Inventive Concept 157. The method according to Inventive Concept 136,
wherein advancing the delivery catheter includes advancing the delivery catheter while the aortic-valve cusp guide, the landing-support stabilizer, and a safety tip of the valve-delivery-assist tool are releasably disposed in the delivery catheter, the safety tip shaped so as to define a guidewire channel through the safety tip, and
wherein the method further includes releasing the safety tip from the distal end of the delivery catheter in the left ventricle such that the safety tip transitions to a deployed shape in the left ventricle.

Inventive Concept 158. The method according to Inventive Concept 157, wherein releasing the safety tip includes releasing the safety tip such that it expands.

Inventive Concept 159. The method according to Inventive Concept 157, wherein the safety tip includes an elongate slender rod.

Inventive Concept 160. The method according to Inventive Concept 159, wherein the elongate slender rod of the released safety tip is configured to be shaped as a pigtail.

The present invention will be more fully understood from the following detailed description of embodiments thereof, taken together with the drawings, in which:

### BRIEF DESCRIPTION OF THE DRAWINGS

FIGS. 1A-C are schematic illustrations of a valve-delivery-assist tool for use during a transcatheter aortic valve implantation (TAVI) procedure on a native aortic valve, in accordance with an application of the present invention;

FIGS. 2A and 2B are schematic illustrations of two respective configurations of another valve-delivery-assist tool for use during a TAVI procedure on a native aortic valve, in accordance with respective applications of the present invention;

FIG. 3 is a schematic illustration of yet another valve-delivery-assist tool for use during a TAVI procedure on a native aortic valve, in accordance with respective applications of the present invention;

FIG. 4 is a schematic illustration of still another valve-delivery-assist tool for use during a TAVI procedure on a native aortic valve, in accordance with respective applications of the present invention;

FIG. 5 is a schematic illustration of another valve-delivery-assist tool for use during a TAVI procedure on a native aortic valve, in accordance with respective applications of the present invention;

FIGS. 6A-N are schematic illustrations of a method of using the valve-delivery-assist tool of FIGS. 1A-C in a TAVI procedure on a native aortic valve, in accordance with an application of the present invention;

FIGS. 7A-7B-2 are schematic illustrations of an alternative way of a deploying an aortic-valve cusp guide and a conduction-tissue protector of the valve-delivery-assist tool of FIGS. 1A-C from a delivery catheter, in accordance with an application of the present invention;

FIG. 8 is a schematic illustration of an alternative way of withdrawing an aortic-valve cusp guide and a conduction-tissue protector of the valve-delivery-assist tool of FIGS. 1A-C into a delivery catheter, in accordance with an application of the present invention;

FIGS. 9A-B are schematic illustrations of a technique for releasing a landing-support stabilizer of the valve-delivery-assist tool of FIGS. 1A-C, in accordance with an application of the present invention;

FIGS. 10A-B are schematic illustrations of another technique for releasing a landing-support stabilizer of the valve-delivery-assist tool of FIGS. 1A-C, in accordance with an application of the present invention;

FIG. 11A-B are schematic illustrations of a configuration of the valve-delivery-assist tool of FIGS. 1A-C further comprising a reshaping element, in accordance with an application of the present invention;

FIGS. 12A-C are schematic illustrations of a contrast-agent-delivery configuration of the valve-delivery-assist tool of FIGS. 1A-C, in accordance with respective applications of the present invention; and

FIG. 13 is a schematic illustration of an embolic protector structure, in accordance with an application of the present invention.

### DETAILED DESCRIPTION OF APPLICATIONS

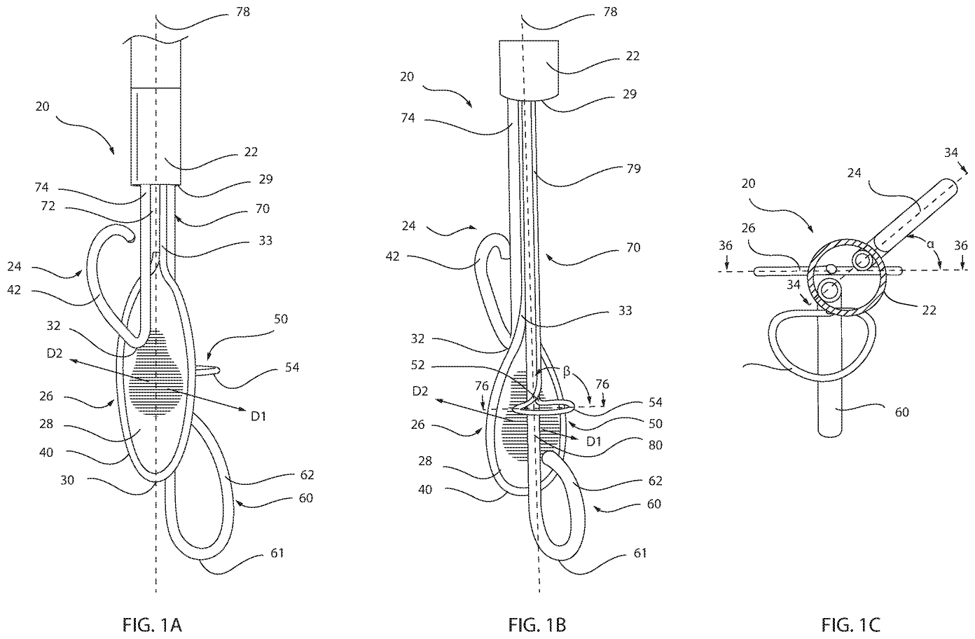

FIGS. 1A-C are schematic illustrations of a valve-delivery-assist tool 20 for use during a transcatheter aortic valve implantation (TAVI) procedure on a native aortic valve, in accordance with an application of the present invention. FIGS. 1A-C show valve-delivery-assist tool 20 upon deployment of an aortic-valve cusp guide 24, a conduction-tissue protector 26, a landing-support stabilizer 50, and a safety tip 60 from a delivery catheter 22. FIG. 1C is a cross-sectional view from a proximal direction.

For some applications, valve-delivery-assist tool 20 comprises:
delivery catheter 22 (which may be flexible and/or steerable);
aortic-valve cusp guide 24, which is releasably disposed in delivery catheter 22; and
conduction-tissue protector 26, which (a) comprises a sheet 28, (b) is releasably disposed compressed in delivery catheter 22, and (c) is configured to expand upon being released from a distal end 29 of delivery catheter 22.

Typically, valve-delivery-assist tool 20 is configured such that aortic-valve cusp guide 24 deflects laterally upon being released from delivery catheter 22, such as described hereinbelow with reference to FIGS. 6F-1 and 6F-2.

For example, aortic-valve cusp guide 24 may comprise a polymer or a shape-memory alloy, such as Nitinol.

Typically, conduction-tissue protector 26 is not shaped so as to define a receptacle (i.e., an internal open space).

Typically, valve-delivery-assist tool 20 is configured such that after lateral deflection of aortic-valve cusp guide 24 and expansion of conduction-tissue protector 26, released conduction-tissue protector 26 and released aortic-valve cusp guide 24 are axially stationary with respect to each other, optionally such that:
a distal-most point 30 of released conduction-tissue protector 26 is disposed distal to a distal-most point 32 of released aortic-valve cusp guide 24, such as between 2 and 30 mm, e.g., between 5 and 10 mm, distal to distal-most point 32 of released aortic-valve cusp guide 24, and/or
a proximal-most point 33 of released conduction-tissue protector 26 is disposed distal to, or no more than 5 mm (e.g., no more than 2 mm) proximal to, distal-most point 32 of released aortic-valve cusp guide 24.

For some applications, such as labeled in FIG. 1C, valve-delivery-assist tool 20 is configured such that a best-fit plane 34 defined by released aortic-valve cusp guide 24 forms an angle a (alpha) of between 15 and 45 degrees (e.g., between 25 and 35 degrees, such as 30 degrees) with a best-fit plane 36 defined by released conduction-tissue protector 26.

For some applications, valve-delivery-assist tool 20 is configured such that after lateral deflection of aortic-valve cusp guide 24 and expansion of conduction-tissue protector 26, released conduction-tissue protector 26 and released aortic-valve cusp guide 24 are rotationally stationary with respect to each other. Alternatively, valve-delivery-assist tool 20 is configured such that after lateral deflection of aortic-valve cusp guide 24 and expansion of conduction-tissue protector 26, released conduction-tissue protector 26 is rotatable with respect to released aortic-valve cusp guide 24. This rotatability allows manual adjustment of conduction-tissue protector 26 with respect to the ventricular wall. This adjustability may be useful in patients whose anatomy differs substantially from more common anatomy for which the standard angle a (alpha) between aortic-valve cusp guide 24 and conduction-tissue protector 26 (described hereinabove with reference to FIG. 1C) is configured in configurations in which aortic-valve cusp guide 24 and conduction-tissue protector 26 are rotationally stationary with respect to each other.

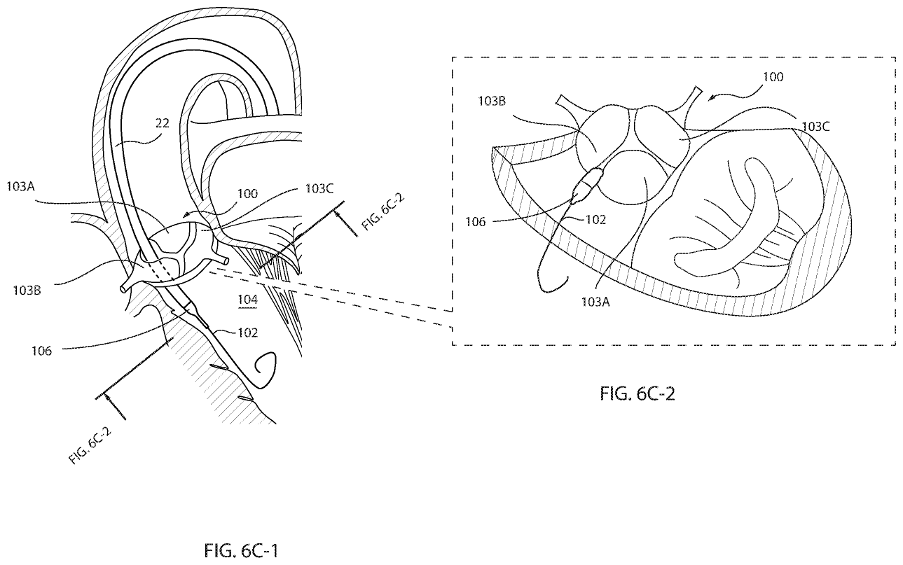

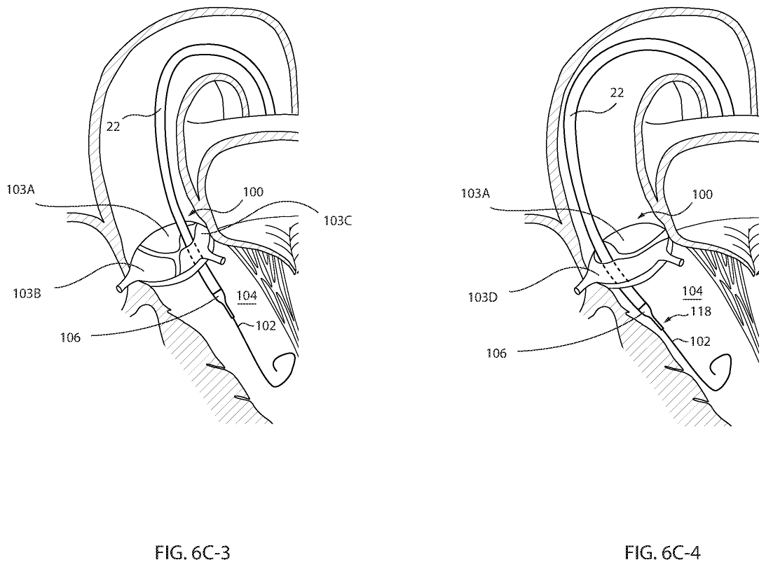

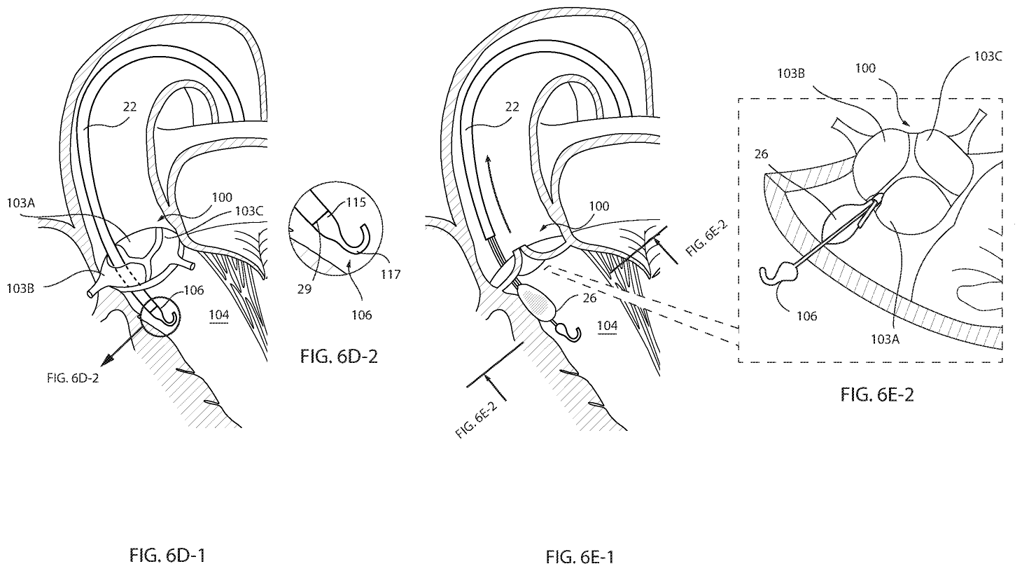

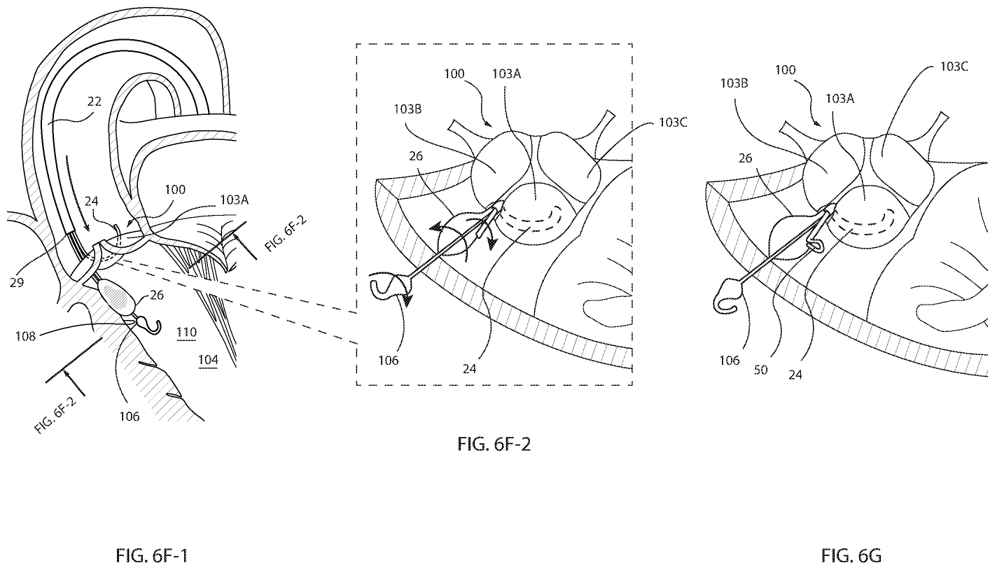

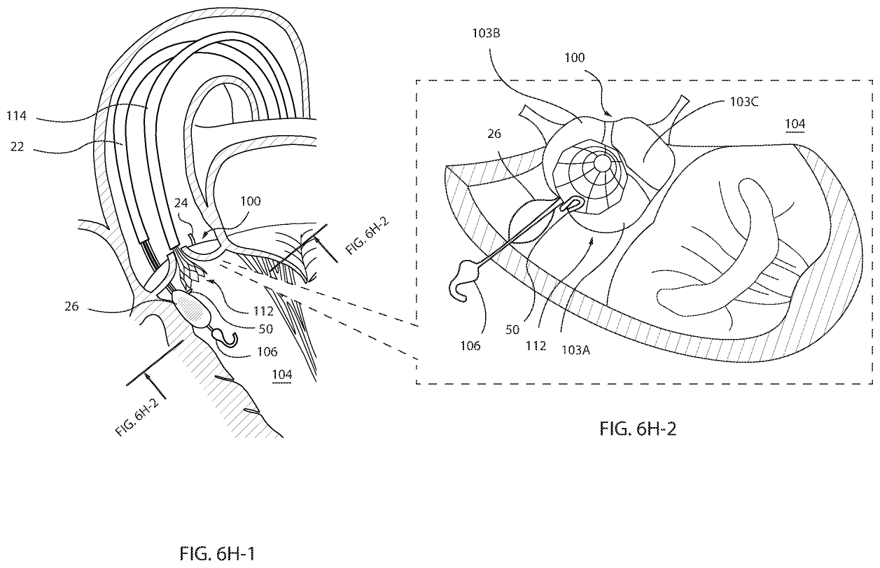

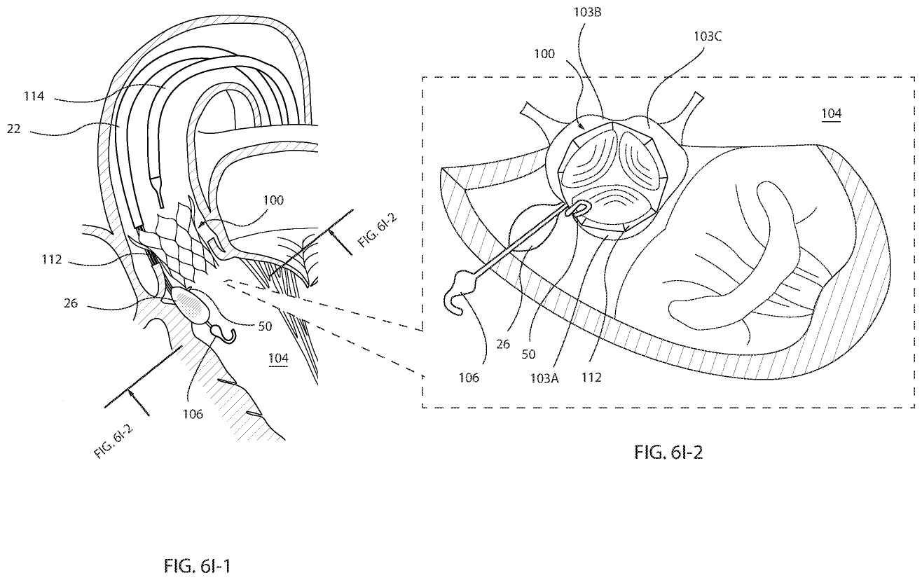

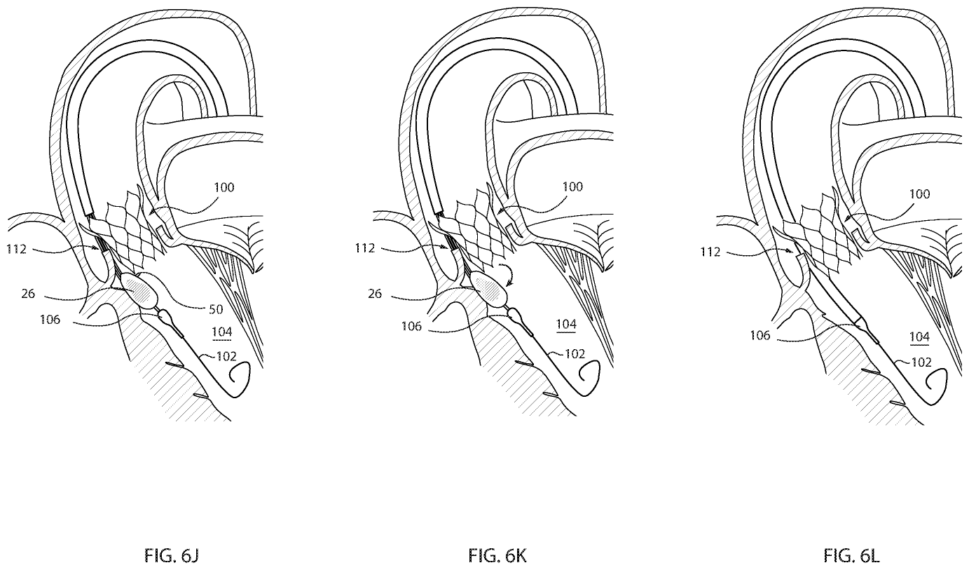

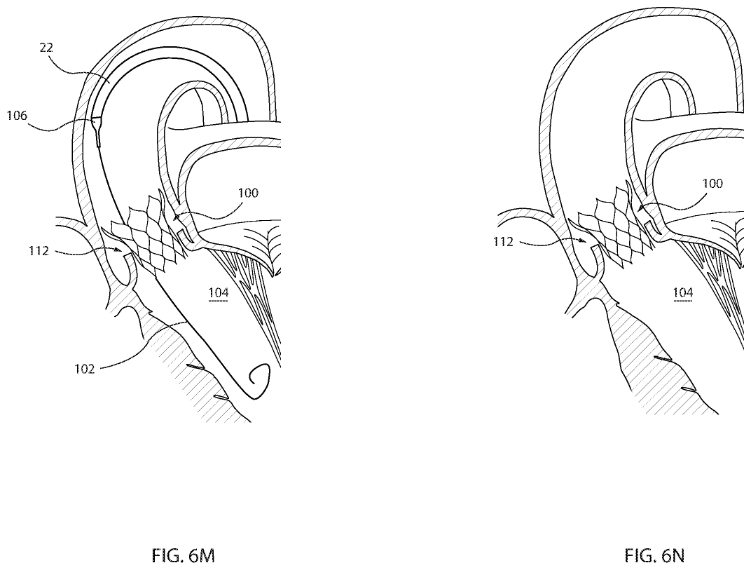

Conduction-tissue protector 26 is configured to distribute mechanical force applied by a prosthetic aortic valve, such as described hereinbelow with reference to FIGS. 6H-K.

For some applications, released conduction-tissue protector 26 is configured such that sheet 28 is generally planar, while for other applications, released conduction-tissue protector 26 is configured such that sheet 28 is not generally planar.

For some applications, sheet 28 has a teardrop shape, an elliptical shape, or a circular shape.

For some applications, sheet 28 comprises a fabric, PTFE, polypropylene, high density polyethylene (PE), polyethylene terephthalate (PET), polytetrafluoroethylene (PTFE), an acellular collagen matrix such as SIS, or another plastic. For other applications, sheet 28 comprises a thin nitinol, stainless steel or titanium sheet. In still other applications, sheet 28 may comprise a combination of materials, such as a high-density PE sheath around a boundary of ePTFE, PET, and/or polypropylene. Sheet 28 may alternatively or additionally comprise polyurethane foam, polycarbonate foam, biologic tissue such as porcine pericardium, bovine pericardium, pleura, peritoneum, and/or silicone. In some applications, sheet 28 may comprise a foam material surrounded by ePTFE. Sheet 28 may be shaped so as not to define any pores, or may be shaped so as to define pores or micropores.

Sheet 28 may or may not be blood-permeable.

For some applications, sheet 28 has:
a surface area of between 1 and 6 cm2; and/or
an average thickness of between 50 and 3000 microns.

For some applications, such as shown in FIGS. 1A-C, conduction-tissue protector 26 further comprises a frame 40 supporting sheet 28. For example, frame 40 may be configured to have a shape memory that is configured to automatically expand sheet 28 to an expanded configuration upon release from delivery catheter 22. For example, frame 40 may comprise one or more wires and/or may comprise a shape-memory alloy, such as Nitinol. Optionally, frame 40 surrounds sheet 28 of released conduction-tissue protector 26, such as shown. Optionally, frame 40 comprises a radiopaque material or an echo-enhancement material for better visualization. Alternatively or additionally, frame 40 optionally may be coated with a radio-opaque material, such as gold or platinum, and/or impregnated with barium.

Frame 40 may be attached to or embedded in the surface of a single layer of sheet 28, or may be stitched through sheet 28.

Alternatively, conduction-tissue protector 26 does not comprise frame 40, in which case sheet 28 provides the necessary structure to maintain the shape of conduction-tissue protector 26 upon deployment. For example, sheet 28 may comprise a polymer that is thick enough to define and maintain the shape.

For some applications, aortic-valve cusp guide 24 comprises an elongate slender rod 42. For example, elongate slender rod 42 may comprise a wire or a tube. For some applications, elongate slender rod 42 of released aortic-valve cusp guide 24 is configured to be shaped as a pigtail (as shown). Optionally, for applications in which elongate slender rod 42 comprises a tube, the tube may be used for introducing a liquid, such as contrast media, during the procedure.

For some applications, valve-delivery-assist tool 20 further comprises landing-support stabilizer 50, which is releasably disposed compressed in delivery catheter 22 and configured to expand (e.g., deflect laterally) after being released from distal end 29 of delivery catheter 22. Landing-support stabilizer 50 may be configured to automatically expand upon being released from distal end 29 of delivery catheter 22, or may be otherwise expanded, such as using one of the techniques described hereinbelow with reference to FIGS. 9A-B or 10A-B. Typically, valve-delivery-assist tool 20 is configured such that after lateral deflection of aortic-valve cusp guide 24 and expansion of conduction-tissue protector 26 and landing-support stabilizer 50: released landing-support stabilizer 50, released conduction-tissue protector 26, and released aortic-valve cusp guide 24 are axially stationary with respect to one another, optionally such that a proximal-most point 52 of released landing-support stabilizer 50 is disposed (a) distal to (e.g., between 1 and 10 mm distal to, such as between 1.5 and 4 mm distal to, e.g., between 2 and 3 mm distal to) distal-most point 32 of released aortic-valve cusp guide 24 and (b) proximal to distal-most point 30 of released conduction-tissue protector 26.

For some applications, valve-delivery-assist tool 20 is configured such that after lateral deflection of aortic-valve cusp guide 24 and expansion of landing-support stabilizer 50, released landing-support stabilizer 50 and released aortic-valve cusp guide 24 are rotationally stationary with respect to one another.

For some applications, valve-delivery-assist tool 20 is configured such that after lateral deflection of aortic-valve cusp guide 24 and expansion of conduction-tissue protector 26 and landing-support stabilizer 50: released landing-support stabilizer 50, released conduction-tissue protector 26, and released aortic-valve cusp guide 24 are rotationally stationary with respect to one another.

For some applications, valve-delivery-assist tool 20 is configured such that after lateral deflection of aortic-valve cusp guide 24 and expansion of conduction-tissue protector 26, released landing-support stabilizer 50 and released conduction-tissue protector 26 are rotationally stationary with respect to one another.

For some applications, landing-support stabilizer 50 comprises an elongate slender rod 54. For example, elongate slender rod 54 may comprise a wire or a tube. For some applications, elongate slender rod 54 of released landing-support stabilizer 50 is shaped as a partial loop or a complete loop having one or more turns.

For some applications, valve-delivery-assist tool 20 is configured such that released landing-support stabilizer 50 and released aortic-valve cusp guide 24 are disposed on opposite sides of released conduction-tissue protector 26.

For some applications, valve-delivery-assist tool 20 further comprises safety tip 60 for assisting with crossing the native valve and providing ventricular access. Safety tip 60 is releasably disposed compressed in delivery catheter 22, and configured to be released from distal end 29 of delivery catheter 22. Typically, safety tip 60 is configured to expand (e.g., deflect laterally) upon released from distal end 29 of delivery catheter 22. Typically, safety tip 60, conduction-tissue protector 26, and aortic-valve cusp guide 24 are linked to one another such that after expansion of conduction-tissue protector 26, a distal-most point 61 of safety tip 60 is disposed distal to distal-most point 30 of released conduction-tissue protector 26. For some applications, safety tip 60 comprises an elongate slender rod 62 (comprising, for example, a wire or a tube), which optionally is configured to be shaped as a pigtail (as shown). (It is noted that distal-most point 61 of safety tip 60 is not necessarily at a distal end of elongate slender rod 62, such as in configurations in which elongate slender rod 62 curves around in a proximal direction.) Optionally, for applications in which elongate slender rod 62 comprises a tube, the tube may be used for introducing a liquid, such as contrast media, during the procedure. For example, safety tip 60 may comprise a polymer.

Safety tip 60 is shaped so as to define a guidewire channel through safety tip 60, for advancement of valve-delivery-assist tool 20 over a guidewire, such as described hereinbelow with reference to FIGS. 6A-C. The guidewire channel may also be used for injecting a liquid, such as contrast media. For example, safety tip 60 may have an outer diameter of 6 Fr.

For some applications, valve-delivery-assist tool 20 further comprises an elongate valve-delivery-assist support 70, which links together conduction-tissue protector 26 and aortic-valve cusp guide 24. Typically, released aortic-valve cusp guide 24 extends laterally from elongate valve-delivery-assist support 70.

For some applications, valve-delivery-assist tool 20 is configured such that released conduction-tissue protector 26 extends from elongate valve-delivery-assist support 70 in two generally opposite directions D1 and D2.

For some applications, valve-delivery-assist tool 20 is configured such that an entirety of released conduction-tissue protector 26 is rotationally fixed with respect to elongate valve-delivery-assist support 70.

For some applications, elongate valve-delivery-assist support 70 comprises:
an elongate conduction-tissue-protector shaft 72, which is fixed to and supports released conduction-tissue protector 26; and
an elongate aortic-valve cusp-guide shaft 74, which is fixed to and supports released aortic-valve cusp guide 24.

Typically, elongate conduction-tissue-protector shaft 72 and elongate aortic-valve cusp-guide shaft 74 are fixed together at least partially alongside each other.

For some applications in which valve-delivery-assist tool 20 comprises landing-support stabilizer 50, elongate valve-delivery-assist support 70 links together landing-support stabilizer 50, conduction-tissue protector 26, and aortic-valve cusp guide 24. For some of these applications, released landing-support stabilizer 50 extends laterally from elongate valve-delivery-assist support 70. For some of these applications, released landing-support stabilizer 50 extends laterally 3-20 mm, such as 3-5 mm, from a central longitudinal axis 78 of elongate valve-delivery-assist support 70 that is straight and passes through the distal end of elongate valve-delivery-assist support 70.

For some of these applications, such as labeled in FIG. 1B, a best-fit plane 76 defined by released landing-support stabilizer 50 forms an angle β (beta) of between 45 and 90 degrees (e.g., 75 and 90 degrees) with central longitudinal axis 78 of elongate valve-delivery-assist support 70.

For some applications in which valve-delivery-assist tool 20 comprises landing-support stabilizer 50, elongate valve-delivery-assist support 70 comprises:
elongate conduction-tissue-protector shaft 72, which is fixed to and supports conduction-tissue protector 26;
elongate aortic-valve cusp-guide shaft 74, which is fixed to and supports aortic-valve cusp guide 24; and
an elongate landing-support-stabilizer shaft 79 (shown in FIG. 1B), which is fixed to and supports landing-support stabilizer 50.

In these applications, typically elongate conduction-tissue-protector shaft 72, elongate aortic-valve cusp-guide shaft 74, and elongate landing-support-stabilizer shaft 79 are fixed together at least partially alongside one another.

For some of these applications, released landing-support stabilizer 50 extends laterally from elongate landing-support-stabilizer shaft 79.

For some of these applications, valve-delivery-assist tool 20 is configured such that best-fit plane 76 defined by released landing-support stabilizer 50 forms an angle of between 45 and 90 degrees (e.g., 75 and 90 degrees) with best-fit plane 36 defined by released conduction-tissue protector 26. For some of these applications, valve-delivery-assist tool 20 is configured such that best-fit plane 34 defined by released aortic-valve cusp guide 24 forms an angle of between 45 and 90 degrees (e.g., 75 and 90 degrees) with best-fit plane 76 defined by released landing-support stabilizer 50.

For some applications in which valve-delivery-assist tool 20 comprises safety tip 60, elongate valve-delivery-assist support 70 comprises, in addition to one or more of the shafts described above, an elongate safety-tip shaft 80 (labeled in FIG. 1B), which is fixed to and supports safety tip 60. For some of these applications, elongate valve-delivery-assist support 70 links together elongate safety-tip shaft 80 and one or more of landing-support stabilizer 50, conduction-tissue protector 26, and aortic-valve cusp guide 24, if provided.

For some of these applications, released safety tip 60 extends laterally from elongate safety-tip shaft 80.

For some applications in which valve-delivery-assist tool 20 does not comprise safety tip 60, elongate valve-delivery-assist support 70 nevertheless comprises, in addition to one or more of the shafts described above, elongate support shaft 80, which may provide structural support to valve-delivery-assist tool 20 and/or aid with deployment of landing-support stabilizer 50, such as described hereinbelow with reference to FIGS. 9A-B or 10A-B. (In this configuration, elongate support shaft 80 is not necessarily referred to as elongate “safety-tip” shaft 80.)

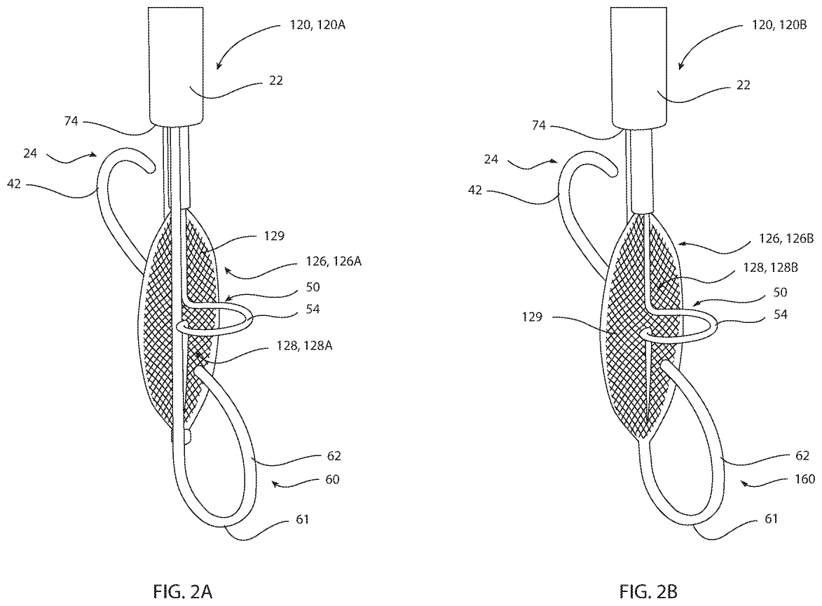

Reference is now made to FIGS. 2A and 2B, which are schematic illustrations of two respective configurations of a valve-delivery-assist tool 120 for use during a TAVI procedure on a native aortic valve, in accordance with respective applications of the present invention. Other than as described below, valve-delivery-assist tool 120 is similar to valve-delivery-assist tool 20, described hereinabove with reference to FIGS. 1A-C, and may implement any of the features thereof, mutatis mutandis. Like reference numerals refer to like parts.

FIGS. 2A and 2B show valve-delivery-assist tool 120 upon deployment of aortic-10 valve cusp guide 24, a conduction-tissue protector 126, landing-support stabilizer 50, and safety tip 60 or 160 from delivery catheter 22.

Valve-delivery-assist tool 120 comprises conduction-tissue protector 126, which comprises a sheet 128 comprising a mesh 129, which is typically flat, such as shown. Mesh 129 typically comprises a metal; for example, mesh 129 may be similar to the mesh of metal stents. For example, mesh 129 may comprise braided wires, such as shown.

Axial elongation of mesh 129 causes radial compression of mesh 129 for placement of conduction-tissue protector 126 with delivery catheter 22. Mesh 129 may provide more stiffness and stability to conduction-tissue protector 126. Mesh 129 may comprise a single layer or two or more layers. In some applications, mesh 129 is covered by a valve body covering such as ePTFE, PET, or porcine pericardium. Mesh is collapsible for introduction through delivery catheter 22.

In the configuration shown in FIG. 2A, valve-delivery-assist tool 120 comprises a valve-delivery-assist tool 120A, and conduction-tissue protector 126 comprises a conduction-tissue protector 126A, which comprises a sheet 128A comprising mesh 129. If provided, safety tip 60 is a separate element from conduction-tissue protector 126A.

In the configuration shown in FIG. 2B, valve-delivery-assist tool 120 comprises a valve-delivery-assist tool 120B, and conduction-tissue protector 126 comprises a conduction-tissue protector 126B, which comprises a sheet 128B comprising mesh 129. Valve-delivery-assist tool 120B further comprises a safety tip 160. Safety tip 160 and conduction-tissue protector 126B are integral to each other. Typically, safety tip 160 comprises a mesh that is an extension of mesh 129.

Although valve-delivery-assist tool 120A is shown in FIG. 2A as comprising landing-support stabilizer 50 and safety tip 60, valve-delivery-assist tool 120A may alternatively comprise neither landing-support stabilizer 50 nor safety tip 60, or only one of landing-support stabilizer 50 and safety tip 60. Although valve-delivery-assist tool 120B is shown in FIG. 2B as comprising landing-support stabilizer 50, valve-delivery-assist tool 120B may alternatively not comprise landing-support stabilizer 50.

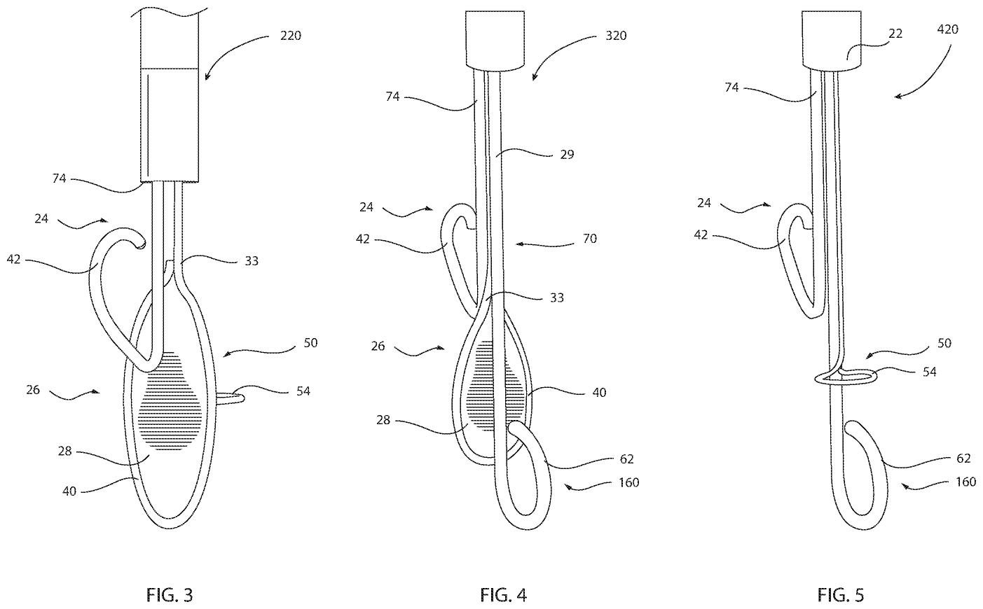

Reference is now made to FIG. 3, which is a schematic illustration of a valve-delivery-assist tool 220 for use during a TAVI procedure on a native aortic valve, in accordance with respective applications of the present invention. Other than as described below, valve-delivery-assist tool 220 is similar to valve-delivery-assist tool 20, described hereinabove with reference to FIGS. 1A-C, and may implement any of the features thereof, mutatis mutandis, and/or any of the features of valve-delivery-assist tool 120, described hereinabove with reference to FIGS. 2A and 2B, mutatis mutandis. Like reference numerals refer to like parts.

Unlike valve-delivery-assist tool 20, valve-delivery-assist tool 220 does not comprise safety tip 60.

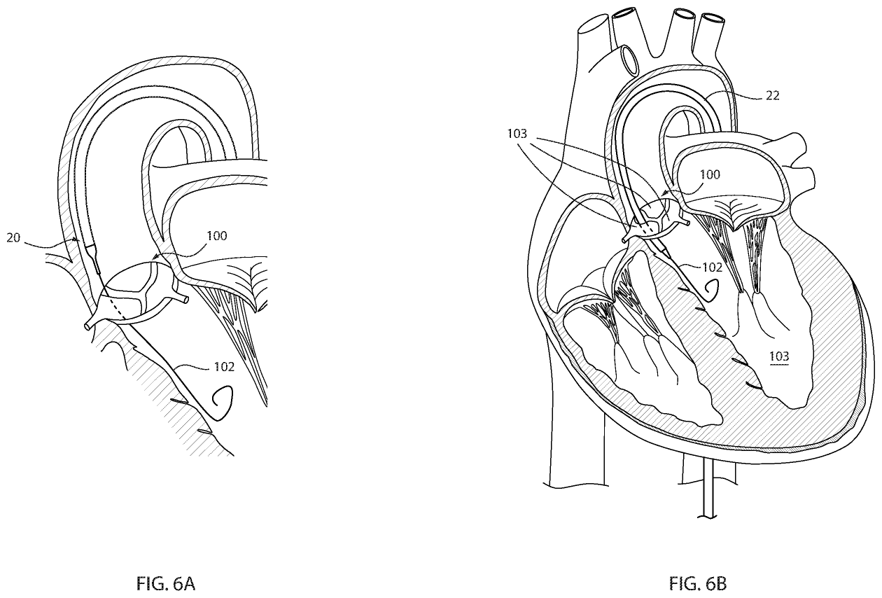

Reference is now made to FIG. 4, which is a schematic illustration of a valve-delivery-assist tool 320 for use during a TAVI procedure on a native aortic valve, in accordance with respective applications of the present invention. Other than as described below, valve-delivery-assist tool 320 is similar to valve-delivery-assist tool 20, described hereinabove with reference to FIGS. 1A-C, and may implement any of the features thereof, mutatis mutandis, and/or any of the features of valve-delivery-assist tool 120, described hereinabove with reference to FIGS. 2A and 2B, mutatis mutandis. Like reference numerals refer to like parts.

Unlike valve-delivery-assist tool 20, valve-delivery-assist tool 320 does not comprise landing-support stabilizer 50.

Reference is now made to FIG. 5, which is a schematic illustration of a valve-delivery-assist tool 420 for use during a TAVI procedure on a native aortic valve, in accordance with respective applications of the present invention. Other than as described below, valve-delivery-assist tool 420 is similar to valve-delivery-assist tool 20, described hereinabove with reference to FIGS. 1A-C, and may implement any of the features thereof, mutatis mutandis. Like reference numerals refer to like parts.

Unlike valve-delivery-assist tool 20, valve-delivery-assist tool 420 does not comprise conduction-tissue protector 26.

Reference is now made to FIGS. 6A-N, which are schematic illustrations of a method of using valve-delivery-assist tool 20 in a TAVI procedure on a native aortic valve 100, in accordance with an application of the present invention. The method may also be performed using valve-delivery-assist tool 20, 120, or 22, described hereinabove with reference to FIGS. 2A-B, 3, and 4, respectively, mutatis mutandis. FIGS. 6A-B, 6C-1, 6C-3, 6D-1, 6E-1, 6F-1, 6H1, 61-1, and 6J-N are cross-sectional views of the heart, and FIGS. 6C-2, 6E-2, 6F-2, 6G, 6H-2, and 61-2 are views of the heart from the ventricles.

As shown in FIGS. 6A-B, valve-delivery-assist tool 20 is introduced over a guidewire 102 into the aorta and advanced to the aortic root, while aortic-valve cusp guide 24, conduction-tissue protector 26, landing-support stabilizer 50, and safety tip 60, to the extent that valve-delivery-assist tool 20 comprises these components, are releasably disposed in delivery catheter 22. For example, femoral arterial access may be used, as is known in the art. Guidewire 102 is advanced between aortic valve cusps 103 into a left ventricle 104, and delivery catheter 22 is advanced over the guidewire.

As shown in FIGS. 6B to FIGS. 6C-1, 6C-2, 6C-3, and 6C-4, delivery catheter 22 is partially advanced into left ventricle, such that at least a nose cone 106 removably coupled to delivery catheter 22 pass through aortic valve cusps 103 into left ventricle 104. Delivery catheter 22 may be passed between any two of aortic valve cusps 103. For example, delivery catheter 22 may be passed between:
a non-coronary cusp (NCC) 103A and a right coronary cusp (RCC) 103B, as shown in FIG. 6C-1 and 6C-2 (and all of the other figures except FIGS. 6C-3 and 6C-4),
NCC 103A and a left coronary cusp (LCC) 103C, as shown in FIG. 6C-3,
LCC 103C and RCC 103B (configuration not shown), or
NCC 103A and a fused left and right coronary cusp 103D of a bicuspid aortic valve, as shown in FIG. 6C-4.

Delivery catheter 22 typically becomes seated in a commissure between two of the cusps.

As shown in FIG. 6D-1 and the blow-up in FIG. 6D-2, guidewire 102 is proximally withdrawn from nose cone 106. For some applications, such as shown in FIGS. 6A-M, nose cone 106 is shaped so as to define (a) a wider proximal base 115 that is removably coupled to distal end 29 of delivery catheter 22, and (b) a narrower elongate distal safety tip 117 that extends distally from proximal base 115. Nose cone 106 is shaped so as to define a guidewire channel through proximal base 115 and elongate distal safety tip 117. Elongate distal safety tip 117 is configured to assume (for example, the tip may comprise a flexible material having a shape memory):
a straight shape 118 when guidewire 102 is disposed in the guidewire channel, such as shown in FIGS. 6A-C and 6J-M, and
a curved shape 119, such as a pigtail shape, when guidewire 102 is not disposed in the guidewire channel, such as shown in FIGS. 6D-I.

Curved shape 119 of nose cone 106 may reduce the risk of perforation of the ventricular wall, including if delivery catheter 22 is advanced farther into the ventricle, such as to the apex of the ventricle. In this configuration, valve-delivery-assist tool 20 typically does not comprise safety tip 60.

For other applications, nose cone 106 is a catheter nose cone known in the art, such as a conventional catheter nose cone. For some of these other applications, valve-delivery-assist tool 20 comprises safety tip 60, such as described hereinabove with reference to FIGS. 1A-C, 2A-B, 4, and 5. Safety tip 60, while constrained within delivery catheter 22, is passed into left ventricle 104, and delivery catheter 22 is moved proximally with respect to safety tip 60 (the catheter is withdrawn proximally and/or the tip is advanced distally), allowing safety tip 60 to transition to a deployed shape in left ventricle 104, such as a pigtail shape (configuration not shown in FIGS. 6A-N).

As shown in FIGS. 6E-1 and 6E-2, delivery catheter 22 is proximally withdrawn, releasing conduction-tissue protector 26, which transitions to a deployed shape in left ventricle 104. Conduction-tissue protector 26 temporarily protects, by mechanically shielding, conduction tissue of the AV node from mechanical compression of the left ventricular outflow tract (LVOT) by a prosthetic aortic valve 112 during the TAVI procedure.

As shown in FIGS. 6F-1 and 6F-2, aortic-valve cusp guide 24 is released from distal end 29 of delivery catheter 22, either by proximally withdrawing delivery catheter 22 (not shown in FIGS. 6F-1 and 6F-2) and/or distally advancing aortic-valve cusp guide 24 (such as shown in FIGS. 6F-1 and 6F-2). Upon release, aortic-valve cusp guide 24 transitions to a deployed shape in the ascending aorta and is seated (typically self-seated) in one of the cusps, typically NCC 103A. Alternatively, aortic-valve cusp guide 24 is manually rotated to help seating in the cusp. Typically, valve-delivery-assist tool 20 is configured such that disposition of released aortic-valve cusp guide 24 into NCC 103A a non-coronary cusp (typically, with the belly of a pigtail of aortic-valve cusp guide 24 on the floor of the NCC and the end of the pigtail on the lateral wall of the cusp) and rotational centering (typically self-centering) of aortic-valve cusp guide 24 within NCC 103A, axially and rotationally align released conduction-tissue protector 26 to face a septal wall 108 of a left ventricular outflow tract (LVOT) 110. This placement causes conduction-tissue protector 26 to indirectly mechanically protect the AV node, thereby reducing the risk of left bundle branch block (LBBB). Typically, aortic-valve cusp guide 24 self-centers within NCC 103A when placed in the NCC. The cusp also prevents further distal advancement of aortic-valve cusp guide 24.

Optionally, aortic-valve cusp guide 24, once seated within the cusp, may also be used to identify the position of native aortic valve 100 and/or the cusp, such as using fluoroscopy.

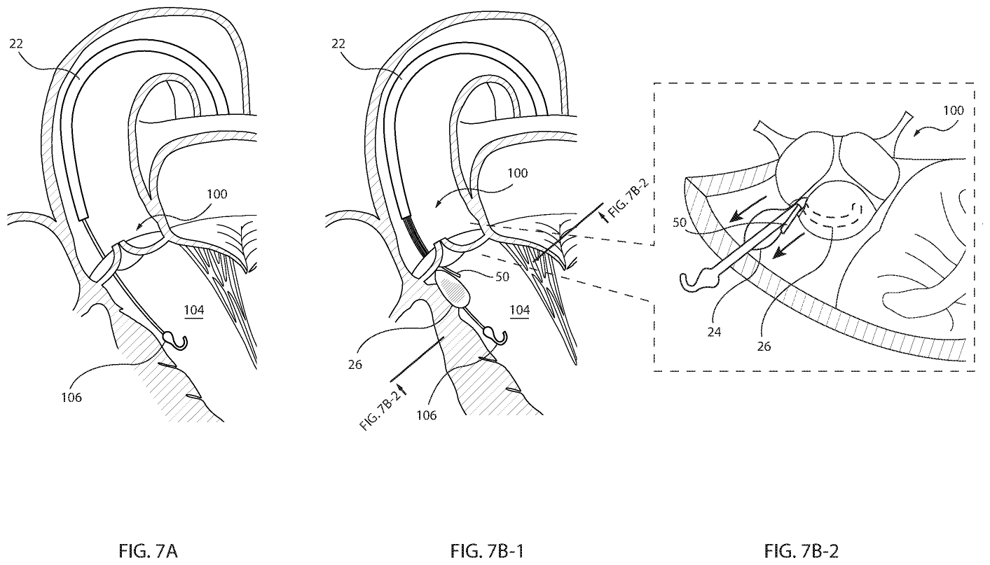

Alternatively, aortic-valve cusp guide 24 and conduction-tissue protector 26 are deployed from delivery catheter 22 as described hereinbelow with reference to FIGS. 7A-7B-2.

As shown in FIG. 6G, landing-support stabilizer 50 is expanded in left ventricle 104, such as using one of the techniques described hereinbelow with reference to FIGS. 9A-B or 10A-B. Typically, valve-delivery-assist tool 20 is configured such that disposition of released aortic-valve cusp guide 24 within NCC 103A and rotationally centering aortic-valve cusp guide 24 within the NCC, axially disposes released landing-support stabilizer 50 at a predetermined height below native aortic valve 100, typically between 2 and 10 mm. The predetermined height serves to define the depth of the subsequent TAVI implantation of a prosthetic aortic valve 112, as described immediately hereinbelow with reference to FIGS. 6H-I.

As shown in FIGS. 6H-1 and 6H-2 to FIGS. 61-1 and 61-2, prosthetic aortic valve 112 is deployed, typically using TAVI techniques known in the art. Typically, a delivery catheter 114 separate from delivery catheter 22 is used for the deployment. Conduction-tissue protector 26 protects, by mechanically shielding, conduction tissue of the AV node from mechanical compression of the LVOT by prosthetic aortic valve 112, and/or from impingement, damage, lesion, abrasion, and/or trauma caused by the metal frame of prosthetic aortic valve 112 on the conductive tissue. Landing-support stabilizer 50 serves to limit the depth of implantation of prosthetic aortic valve 112 to the predetermined height. This depth limiting may reduce the risk of paravalvular leakage (PVL) that may be caused by inappropriate depth of implantation.

FIG. 6J shows prosthetic aortic valve 112 upon completion of deployment, before the withdrawal of valve-delivery-assist tool 20.

As shown in FIG. 6K, landing-support stabilizer 50 is retracted.

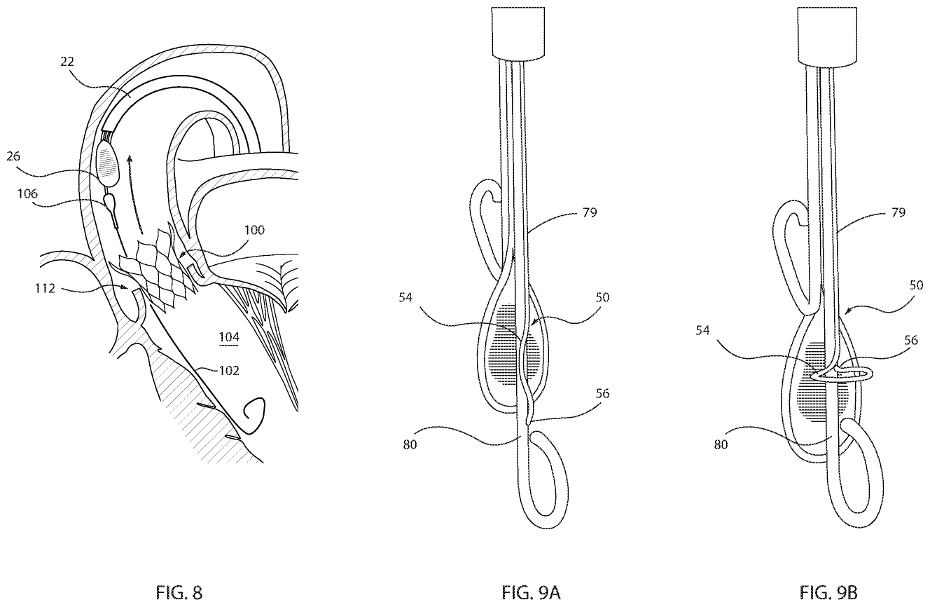

As shown in FIG. 6L, aortic-valve cusp guide 24 and conduction-tissue protector 26 are withdrawn into delivery catheter 22, such as by proximally advancing the delivery catheter over the cusp guide and protector, as shown. Alternatively, aortic-valve cusp guide 24 and conduction-tissue protector 26 are withdrawn into delivery catheter 22 as described hereinbelow with reference to FIG. 8.

As shown in FIG. 6M, delivery catheter 22 is withdrawn from native aortic valve 100, leaving prosthetic aortic valve 112 implanted in the native aortic valve, as shown in FIG. 6N.

Reference is now made to FIGS. 7A-7B-2, which are schematic illustrations of an alternative way of deploying aortic-valve cusp guide 24 and conduction-tissue protector 26 from delivery catheter 22, in accordance with an application of the present invention. This deployment technique is optionally used instead of the deployment technique described hereinabove with reference to FIGS. 6E-F.

As shown in FIG. 7A, while nose cone 106 remains in left ventricle 104, delivery catheter 22 is proximally withdrawn in the ascending aorta, while aortic-valve cusp guide 24 and conduction-tissue protector 26 remain axially stationary with respect to delivery catheter 22.

As shown in FIGS. 7B-1 and 7B-2, aortic-valve cusp guide 24 and conduction-tissue protector 26 are advanced distally out of delivery catheter 22, while the delivery catheter remains axially stationary with respect to the ascending aorta.

Reference is now made to FIG. 8, which is a schematic illustration of an alternative way of withdrawing aortic-valve cusp guide 24 and conduction-tissue protector 26 into delivery catheter 22, in accordance with an application of the present invention. This deployment technique is optionally used instead of the deployment technique described hereinabove with reference to FIG. 6L. As shown in FIG. 8, aortic-valve cusp guide 24 and conduction-tissue protector 26 are proximally withdrawn through a commissure of native aortic valve 100 outside of prosthetic aortic valve 112, while aortic-valve cusp guide 24 and conduction-tissue protector 26 are not sheathed by delivery catheter 22. Subsequently, delivery catheter 22 is advanced distally over aortic-valve cusp guide 24 and conduction-tissue protector 26 (step not shown).

Reference is now made to FIGS. 9A-B, which are schematic illustrations of a technique for manually expanding landing-support stabilizer 50, in accordance with an application of the present invention. In this configuration of valve-delivery-assist tool 20, a distal end 56 of elongate slender rod 54 of landing-support stabilizer 50 is fixed to elongate support shaft 80. Disposal of elongate support shaft 80 in a more distal position, such as shown in FIG. 9A, stretches elongate slender rod 54 and radially compresses it against elongate support shaft 80. Disposal of elongate support shaft 80 in a more proximal position, such as shown in FIG. 9B, causes elongate slender rod 54 to bulge away from, and radially expand with respect to, elongate support shaft 80, such as to become shaped as a partial loop or a complete loop having one or more turns.

Reference is now made to FIGS. 10A-B, which are schematic illustrations of another technique for manually expanding landing-support stabilizer 50, in accordance with an application of the present invention. In this configuration of valve-delivery-assist tool 20, elongate support shaft 80 is shaped so as to define a channel having a distal opening 82 through which elongate slender rod 54 of landing-support stabilizer 50 is slidable. Withdrawal of elongate slender rod 54 into the channel, such as shown in FIG. 10A, radially compresses the elongate slender rod within elongate support shaft 80. Extension of elongate slender rod 54 from the channel, such as shown in FIG. 10B, causes elongate slender rod 54 to bulge away from, and radially expand with respect to, elongate support shaft 80, such as to become shaped as a partial loop or a complete loop having one or more turns. Thus, in this configuration, valve-delivery-assist tool 20 is configured such that landing-support stabilizer 50 expands after being released from delivery catheter 22 (and after the subsequent extension of elongate slender rod 54 from the channel), rather than immediately upon release of landing-support stabilizer 50 from distal end 29 of delivery catheter 22. Optionally, elongate support shaft 80 is further configured such that its channel additionally serves as a guidewire channel through which guidewire 102 passes.

Reference is still made to FIGS. 10A-B. In an application of the present invention, the techniques of FIGS. 10A-B are implemented mutatis mutandis for deploying aortic-valve cusp guide 24. In this configuration, valve-delivery-assist tool 20 comprises an elongate support shaft that is shaped so as to define a channel having a distal opening through which elongate slender rod 42 of aortic-valve cusp guide 24 is slidable. Withdrawal of elongate slender rod 42 into the channel, in a manner similar to that shown in FIG. 10A for elongate slender rod 54 of landing-support stabilizer 50, radially compresses elongate slender rod 42 within the elongate support shaft. Extension of elongate slender rod 42 from the channel, in a manner similar to that shown in FIG. 10B for elongate slender rod 54 of landing-support stabilizer 50, causes elongate slender rod 42 to bulge away from, and radially expand with respect to, the elongate support shaft. Thus, in this configuration, valve-delivery-assist tool 20 is configured such that aortic-valve cusp guide 24 deflects laterally after being released from delivery catheter 22 (and after the subsequent extension of elongate slender rod 42 from the channel), rather than immediately upon release of aortic-valve cusp guide 24 from delivery catheter 22. Optionally, the elongate support shaft is further configured such that its channel additionally serves as a guidewire channel through which guidewire 102 passes.

In an application of the present invention, valve-delivery-assist tool 20 comprises a protector delivery shaft. Conduction-tissue protector 26 is releasably disposed compressed in the protector delivery shaft, and is configured to expand upon being released from the protector delivery shaft. The protector delivery shaft is slidably disposed within delivery catheter 22, such that conduction-tissue protector is releasably disposed compressed in delivery catheter 22 (indirectly, via the protector delivery shaft). Thus, in this configuration, valve-delivery-assist tool 20 is configured such that conduction-tissue protector 26 expands after being released from both distal end 29 of delivery catheter and from the protector delivery shaft, rather than immediately upon release of conduction-tissue protector 26 from distal end 29 of delivery catheter 22.

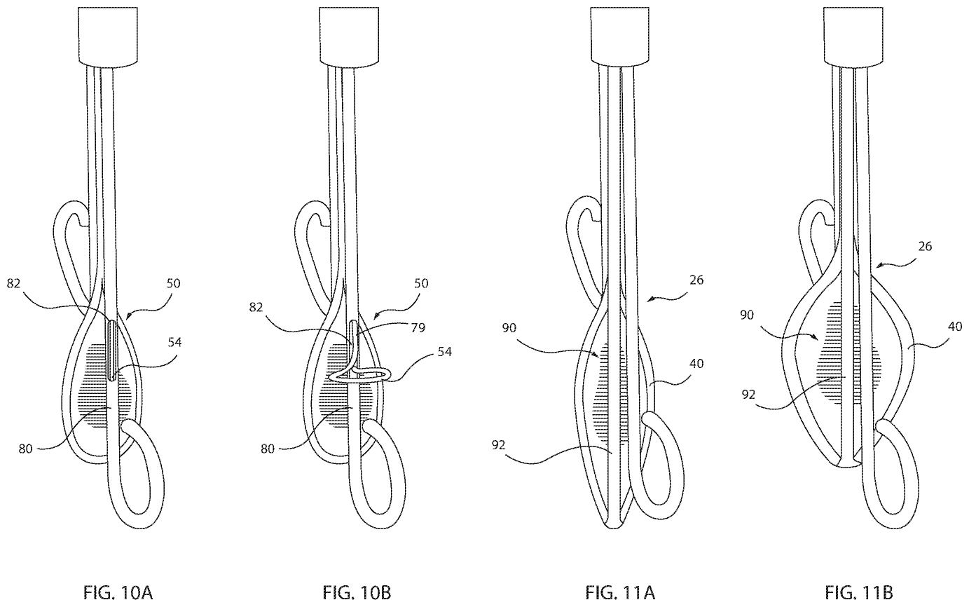

Reference is now made to FIG. 11A-B, which are schematic illustrations of a configuration of valve-delivery-assist tool 20 further comprising a reshaping element 90, in accordance with an application of the present invention. Reshaping element 90 can be implemented in combination with any of the valve-delivery-assist tools and configurations thereof described herein, mutatis mutandis.

Reshaping element 90 is coupled to conduction-tissue protector 26, such as to frame 40 of conduction-tissue protector 26, e.g., a distal portion of the frame or a distal portion of another element of conduction-tissue protector 26.

Reshaping element 90 is configured to change a shape of released conduction-tissue protector 26. For example, reshaping element 90 may be configured to change a vertical-axis dimension of released conduction-tissue protector 26, such as by axially shortening released conduction-tissue protector 26, as shown in the transition between FIG. 11A and FIG. 11B. The change in shape typically does not change the axial relationships among the components of valve-delivery-assist tool 20.

For some applications, reshaping element 90 comprises an elongate reshaping shaft 92. Pulling or pushing on elongate reshaping shaft 92 causes the change in shape of released conduction-tissue protector 26.

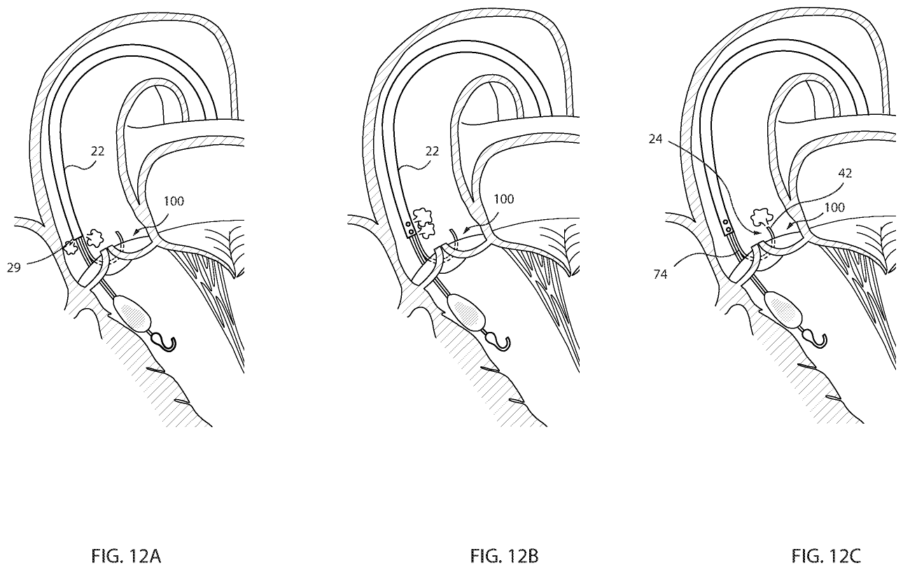

Reference is now made to FIGS. 12A-C, which are schematic illustrations of a contrast-agent-delivery configuration of valve-delivery-assist tool 20, in accordance with respective applications of the present invention. For any of the applications described and/or shown herein, valve-delivery-assist tool 20 may optionally be shaped so to define a contrast channel, which is shaped so as to define one or more lateral or distal openings for injection of contrast media (or another substance, such as saline solution). For some applications, delivery catheter 22 is shaped so as to define the contrast channel (either by an independent shaft or tube that is internal to delivery catheter 22, such as shown in FIG. 12B, optionally one or more lateral openings through a wall of the delivery catheter, or directly using the internal channel of delivery catheter 22, such as shown in FIG. 12A, such as via distal end 29 of delivery catheter 22). For other applications, one or more of the elongate shafts and/or elongate slender rods described herein is shaped so as to define the contrast channel, such as shown by way of example and not limitation in FIG. 12C for elongate slender rod 42 of aortic-valve cusp guide 24 and elongate aortic-valve cusp-guide shaft 74.

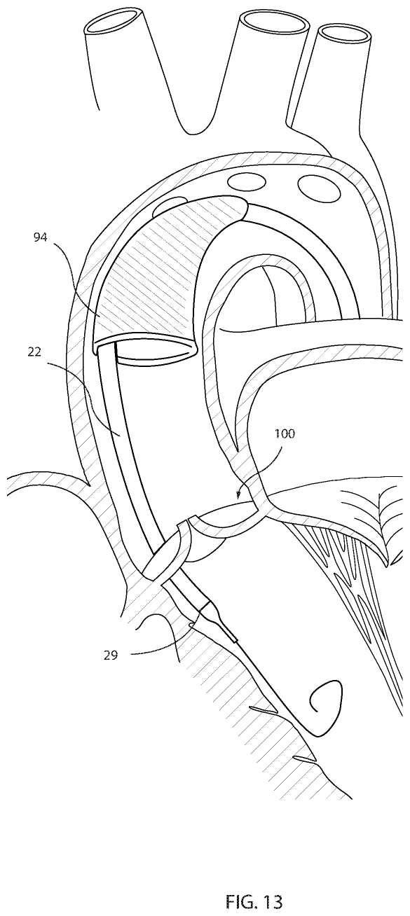

Reference is now made to FIG. 13, which is a schematic illustration of an embolic protector structure 94, in accordance with an application of the present invention. For any of the applications described and/or shown herein, delivery catheter 22 may optionally comprise embolic protector structure 94, which comprises a filter or mesh, which can be elongated and opened proximally to distal end 29 of delivery catheter 22.

In an embodiment, techniques and apparatus described in U.S. Provisional Application 63/070,300, filed Aug. 26, 2020, including, but not limited to the Drawings attached thereto, are combined with techniques and apparatus described herein.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A valve-delivery-assist tool for use during a transcatheter aortic valve implantation (TAVI) procedure on a native aortic valve, the valve-delivery-assist tool comprising:
a delivery catheter;
an aortic-valve cusp guide, which is releasably disposed in the delivery catheter, wherein the valve-delivery-assist tool is configured such that the aortic-valve cusp guide deflects laterally upon being released from the delivery catheter; and
a conduction-tissue protector, which (a) comprises a sheet, (b) is releasably disposed compressed in the delivery catheter, and (c) is configured to expand upon being released from a distal end of the delivery catheter,
wherein the valve-delivery-assist tool is configured such that after lateral deflection of the aortic-valve cusp guide and expansion of the conduction-tissue protector, the released conduction-tissue protector and the released aortic-valve cusp guide are axially stationary with respect to each other such that a distal-most point of the released conduction-tissue protector is disposed distal to a distal-most point of the released aortic-valve cusp guide.

2. The valve-delivery-assist tool according to claim 1, wherein the valve-delivery-assist tool is configured such that disposition of the released aortic-valve cusp guide into a non-coronary cusp of the native aortic valve and rotational centering of the aortic-valve cusp guide within the non-coronary cusp, axially and rotationally align the released conduction-tissue protector to face a septal wall of a left ventricular outflow tract (LVOT).

3. The valve-delivery-assist tool according to claim 1, wherein the valve-delivery-assist tool is configured such that after lateral deflection of the aortic-valve cusp guide and expansion of the conduction-tissue protector, the released conduction-tissue protector and the released aortic-valve cusp guide are axially stationary with respect to each other such that the distal-most point of the released conduction-tissue protector is disposed between 2 and 30 mm distal to the distal-most point of the released aortic-valve cusp guide.

4. The valve-delivery-assist tool according to claim 1, wherein the valve-delivery-assist tool is configured such that after lateral deflection of the aortic-valve cusp guide and expansion of the conduction-tissue protector, the released conduction-tissue protector and the released aortic-valve cusp guide are axially stationary with respect to each other such that a proximal-most point of the released conduction-tissue protector is disposed distal to, or no more than 5 mm proximal to, the distal-most point of the released aortic-valve cusp guide.

5. The valve-delivery-assist tool according to claim 1, wherein the valve-delivery-assist tool is configured such that after lateral deflection of the aortic-valve cusp guide and expansion of the conduction-tissue protector, the released conduction-tissue protector and the released aortic-valve cusp guide are rotationally stationary with respect to each other.

6. The valve-delivery-assist tool according to claim 1, wherein the conduction-tissue protector further comprises a frame supporting the sheet.

7. The valve-delivery-assist tool according to claim 6, wherein the frame surrounds the sheet of the released conduction-tissue protector.

8. The valve-delivery-assist tool according to claim 6, wherein the frame comprises one or more wires.

9. The valve-delivery-assist tool according to claim 1, wherein the released conduction-tissue protector is configured such that the sheet is generally planar.

10. The valve-delivery-assist tool according to claim 1, wherein the sheet comprises a mesh.

11. The valve-delivery-assist tool according to claim 10, wherein the mesh comprises braided wires.

12. The valve-delivery-assist tool according to any one of claims 1-11, wherein the aortic-valve cusp guide comprises an elongate slender rod.

13. The valve-delivery-assist tool according to claim 12, wherein the elongate slender rod of the released aortic-valve cusp guide is configured to be shaped as a pigtail.

14. The valve-delivery-assist tool according to any one of claims 1-11, wherein the valve-delivery-assist tool is configured such that a best-fit plane defined by the released aortic-valve cusp guide forms an angle of between 15 and 60 degrees with a best-fit plane defined by the released conduction-tissue protector.

15. The valve-delivery-assist tool according to claim 14, wherein the valve-delivery-assist tool is configured such that the angle is between 25 and 35 degrees.

16. The valve-delivery-assist tool according to any one of claims 1-11, further comprising an elongate valve-delivery-assist support, which links together the conduction-tissue protector and the aortic-valve cusp guide.

17. The valve-delivery-assist tool according to claim 16, wherein the released aortic-valve cusp guide extends laterally from the elongate valve-delivery-assist support.

18. The valve-delivery-assist tool according to claim 16, wherein the valve-delivery-assist tool is configured such that the released conduction-tissue protector extends from the elongate valve-delivery-assist support in two generally opposite directions.

19. The valve-delivery-assist tool according to claim 16, wherein the valve-delivery-assist tool is configured such that an entirety of the released conduction-tissue protector is rotationally fixed with respect to the elongate valve-delivery-assist support.

20. The valve-delivery-assist tool according to claim 16, wherein the elongate valve-delivery-assist support comprises:
an elongate conduction-tissue-protector shaft, which is fixed to and supports the released conduction-tissue protector; and
an elongate aortic-valve cusp-guide shaft, which is fixed to and supports the released aortic-valve cusp guide,
wherein the elongate conduction-tissue-protector shaft and the elongate aortic-valve cusp-guide shaft are fixed together at least partially alongside each other.

21. The valve-delivery-assist tool according to any one of claims 1-11, further comprising a landing-support stabilizer, which is releasably disposed compressed in the delivery catheter and configured to expand after being released from the distal end of the delivery catheter,
wherein the valve-delivery-assist tool is configured such that after lateral deflection of the aortic-valve cusp guide and expansion of the conduction-tissue protector and the landing-support stabilizer:
the released landing-support stabilizer, the released conduction-tissue protector, and the released aortic-valve cusp guide are axially stationary with respect to one another such that a proximal-most point of the released landing-support stabilizer is disposed (a) distal to the distal-most point of the released aortic-valve cusp guide and (b) proximal to the distal-most point of the released conduction-tissue protector.

22. The valve-delivery-assist tool according to claim 21, wherein the landing-support stabilizer is configured to automatically expand upon being released from the distal end of the delivery catheter.

23. The valve-delivery-assist tool according to claim 21, wherein the landing-support stabilizer is configured to be manually expanded after being released from the distal end of the delivery catheter.

24. The valve-delivery-assist tool according to claim 21, wherein the valve-delivery-assist tool is configured such that after lateral deflection of the aortic-valve cusp guide and expansion of the conduction-tissue protector and the landing-support stabilizer, the released landing-support stabilizer, the released conduction-tissue protector, and the released aortic-valve cusp guide are axially stationary with respect to one another such that the proximal-most point of the released landing-support stabilizer is disposed between 1 and 10 mm distal to the distal-most point of the released aortic-valve cusp guide.

25. The valve-delivery-assist tool according to claim 24, wherein the valve-delivery-assist tool is configured such that after lateral deflection of the aortic-valve cusp guide and expansion of the conduction-tissue protector and the landing-support stabilizer, the released landing-support stabilizer, the released conduction-tissue protector, and the released aortic-valve cusp guide are axially stationary with respect to one another such that the proximal-most point of the released landing-support stabilizer is disposed between 1.5 and 4 mm distal to the distal-most point of the released aortic-valve cusp guide.

26. The valve-delivery-assist tool according to claim 21, wherein the valve-delivery-assist tool is configured such that disposition of the released aortic-valve cusp guide within a non-coronary cusp of the native aortic valve and rotationally centering the aortic-valve cusp guide within the non-coronary cusp, axially disposes the released landing-support stabilizer at a predetermined height below the native aortic valve.

27. The valve-delivery-assist tool according to claim 26, wherein the predetermined height is between 2 and 10 mm.

28. The valve-delivery-assist tool according to claim 21, wherein the valve-delivery-assist tool is configured such that after lateral deflection of the aortic-valve cusp guide and expansion of the conduction-tissue protector and the landing-support stabilizer:
the released conduction-tissue protector and the released aortic-valve cusp guide are rotationally stationary with respect to one another.

29. The valve-delivery-assist tool according to claim 28, wherein the valve-delivery-5 assist tool is configured such that after lateral deflection of the aortic-valve cusp guide and expansion of the conduction-tissue protector and the landing-support stabilizer:
the released landing-support stabilizer, the released conduction-tissue protector, and the released aortic-valve cusp guide are rotationally stationary with respect to one another.

30. The valve-delivery-assist tool according to claim 21, wherein the valve-delivery-assist tool is configured such that after lateral deflection of the aortic-valve cusp guide and expansion of the conduction-tissue protector and the landing-support stabilizer:
the released landing-support stabilizer and the released conduction-tissue protector are rotationally stationary with respect to one another.

31. The valve-delivery-assist tool according to claim 21, wherein the valve-delivery-assist tool is configured such that the released landing-support stabilizer and the released aortic-valve cusp guide are disposed on opposite sides of the released conduction-tissue protector.

32. The valve-delivery-assist tool according to claim 21, wherein the landing-support stabilizer comprises an elongate slender rod.

33. The valve-delivery-assist tool according to claim 32, wherein the elongate slender rod of the released landing-support stabilizer is shaped as a partial loop or a complete loop having one or more turns.

34. The valve-delivery-assist tool according to claim 33,
wherein the valve-delivery-assist tool further comprises (a) an elongate landing-support-stabilizer shaft, which is fixed to and supports the landing-support stabilizer, and (b) an elongate support shaft, and
wherein a distal end of the elongate slender rod is fixed to the elongate support shaft such that:
disposal of the elongate support shaft in a more distal position stretches the elongate slender rod and radially compresses the elongate slender rod against the elongate support shaft, and
disposal of the elongate support shaft in a more proximal position causes the elongate slender rod to bulge away from, and radially expand with respect to, the elongate support shaft.

35. The valve-delivery-assist tool according to claim 33,
wherein the valve-delivery-assist tool further comprises (a) an elongate landing-support-stabilizer shaft, which is fixed to and supports the landing-support stabilizer, and (b) an elongate support shaft, which is shaped so as to define a channel having a distal opening through which the elongate slender rod is slidable, and
wherein the valve-delivery-assist tool is configured such that:
withdrawal of the elongate slender rod into the channel radially compresses the elongate slender rod within the elongate support shaft, and
extension of the elongate slender rod from the channel causes the elongate slender rod to bulge away from, and radially expand with respect to, the elongate support shaft.

36. The valve-delivery-assist tool according to claim 21, further comprising an elongate valve-delivery-assist support, which links together the landing-support stabilizer, the conduction-tissue protector, and the aortic-valve cusp guide.

37. The valve-delivery-assist tool according to claim 36, wherein the released landing-support stabilizer extends laterally from the elongate valve-delivery-assist support.

38. The valve-delivery-assist tool according to claim 37, wherein a best-fit plane defined by the released landing-support stabilizer forms an angle of between 45 and 90 degrees with a central longitudinal axis of the elongate valve-delivery-assist support that is straight and passes through the distal end of the elongate valve-delivery-assist support.

39. The valve-delivery-assist tool according to claim 36, wherein the elongate valve-delivery-assist support comprises:
an elongate conduction-tissue-protector shaft, which is fixed to and supports the conduction-tissue protector;
an elongate aortic-valve cusp-guide shaft, which is fixed to and supports the aortic-valve cusp guide; and
an elongate landing-support-stabilizer shaft, which is fixed to and supports the landing-support stabilizer,
wherein the elongate conduction-tissue-protector shaft, the elongate aortic-valve cusp-guide shaft, and the elongate landing-support-stabilizer shaft are fixed together at least partially alongside one another.

40. The valve-delivery-assist tool according to claim 21, wherein the valve-delivery-assist tool is configured such that a best-fit plane defined by the released landing-support stabilizer forms an angle of between 45 and 90 degrees with a best-fit plane defined by the released conduction-tissue protector.

41. The valve-delivery-assist tool according to claim 40, wherein the valve-delivery-assist tool is configured such that a best-fit plane defined by the released aortic-valve cusp guide forms an angle of between 45 and 90 degrees with the best-fit plane defined by the released landing-support stabilizer.

42. The valve-delivery-assist tool according to claim 21,
further comprising a safety tip, which is releasably disposed compressed in the delivery catheter, configured to be released from the distal end of the delivery catheter, and shaped so as to define a guidewire channel through the safety tip,
wherein the safety tip, the conduction-tissue protector, and the aortic-valve cusp guide are linked to one another such that after expansion of the conduction-tissue protector, a distal-most point of the safety tip is disposed distal to the distal-most point of the released conduction-tissue protector.

43. The valve-delivery-assist tool according to claim 42, wherein the safety tip is configured to expand upon released from the distal end of the delivery catheter.

44. The valve-delivery-assist tool according to claim 42, wherein the safety tip comprises an elongate slender rod.

45. The valve-delivery-assist tool according to claim 44, wherein the elongate slender rod of the released safety tip is configured to be shaped as a pigtail.

46. The valve-delivery-assist tool according to claim 42, wherein the safety tip and the conduction-tissue protector are integral to each other.

47. The valve-delivery-assist tool according to any one of claims 1-11, for use with a guidewire,
wherein the valve-delivery-assist tool further comprises a nose cone, which is shaped so as to define (a) a wider proximal base that is removably coupled to a distal end of the delivery catheter, and (b) a narrower elongate distal safety tip that extends distally from the proximal base,
wherein the nose cone is shaped so as to define a guidewire channel through the proximal base and the elongate distal safety tip, and
wherein the elongate distal safety tip is configured to assume a straight shape when the guidewire is disposed in the guidewire channel, and a curved shape when the guidewire is not disposed in the guidewire channel.

48. The valve-delivery-assist tool according to claim 47, wherein the curved shape is a pigtail shape.

49. The valve-delivery-assist tool according to any one of claims 1-11,
further comprising a safety tip, which is releasably disposed compressed in the delivery catheter, configured to be released from the distal end of the delivery catheter, and shaped so as to define a guidewire channel through the safety tip,
wherein the safety tip, the conduction-tissue protector, and the aortic-valve cusp guide are linked to one another such that after expansion of the conduction-tissue protector, a distal-most point of the safety tip is disposed distal to the distal-most point of the released conduction-tissue protector.

50. The valve-delivery-assist tool according to claim 49, wherein the safety tip is configured to expand upon released from the distal end of the delivery catheter.

51. The valve-delivery-assist tool according to claim 49, wherein the safety tip comprises an elongate slender rod.

52. The valve-delivery-assist tool according to claim 51, wherein the elongate slender rod of the released safety tip is configured to be shaped as a pigtail.

53. The valve-delivery-assist tool according to claim 49, wherein the safety tip and the conduction-tissue protector are integral to each other.

54. The valve-delivery-assist tool according to any one of claims 1-11, further comprising a reshaping element, which is coupled to the conduction-tissue protector, which is configured to change a shape of the released conduction-tissue protector.

55. The valve-delivery-assist tool according to claim 54,
wherein the conduction-tissue protector further comprises a frame supporting the sheet, and
wherein the reshaping element is coupled to the frame of the conduction-tissue protector.

56. The valve-delivery-assist tool according to claim 54, wherein the reshaping element comprises an elongate reshaping shaft.

57. The valve-delivery-assist tool according to any one of claims 1-11, wherein the valve-delivery-assist tool is shaped so to define a contrast channel, which is shaped so as to define one or more lateral or distal openings for injection of contrast media.

58. The valve-delivery-assist tool according to claim 57, wherein the delivery catheter is shaped so as to define the contrast channel.

59. The valve-delivery-assist tool according to claim 57, wherein the valve-delivery-assist tool comprises an elongate shaft that is shaped so as to define the contrast channel

60. The valve-delivery-assist tool according to any one of claims 1-11, wherein the delivery catheter comprises an embolic protector structure, which comprises a filter or mesh, and which can be elongated and opened proximally to a distal end of the embolic protector structure.

61. A valve-delivery-assist tool for use during a transcatheter aortic valve implantation (TAVI) procedure on a native aortic valve, the valve-delivery-assist tool comprising:
a delivery catheter;
an aortic-valve cusp guide, which is releasably disposed in the delivery catheter, wherein the valve-delivery-assist tool is configured such that the aortic-valve cusp guide deflects laterally upon being released from the delivery catheter; and
a landing-support stabilizer, which is releasably disposed compressed in the delivery catheter and configured to expand after being released from a distal end of the delivery catheter.

62. The valve-delivery-assist tool according to claim 61, wherein the landing-support stabilizer is configured to automatically expand upon being released from the distal end of the delivery catheter.

63. The valve-delivery-assist tool according to claim 61, wherein the landing-support stabilizer is configured to be manually expanded after being released from the distal end of the delivery catheter.

64. The valve-delivery-assist tool according to claim 61, wherein the valve-delivery-assist tool is configured such that after lateral deflection of the aortic-valve cusp guide and expansion of the landing-support stabilizer, the released landing-support stabilizer and the released aortic-valve cusp guide are axially stationary with respect to one another such that a proximal-most point of the released landing-support stabilizer is disposed distal to the distal-most point of the released aortic-valve cusp guide.

65. The valve-delivery-assist tool according to claim 64, wherein the valve-delivery-assist tool is configured such that after lateral deflection of the aortic-valve cusp guide and expansion of the landing-support stabilizer, the released landing-support stabilizer and the released aortic-valve cusp guide are axially stationary with respect to one another such that the proximal-most point of the released landing-support stabilizer is disposed between 1 and 10 mm distal to the distal-most point of the released aortic-valve cusp guide.

66. The valve-delivery-assist tool according to claim 65, wherein the valve-delivery-assist tool is configured such that after lateral deflection of the aortic-valve cusp guide and expansion of the landing-support stabilizer, the released landing-support stabilizer and the released aortic-valve cusp guide are axially stationary with respect to one another such that the proximal-most point of the released landing-support stabilizer is disposed between 1.5 and 4 mm distal to the distal-most point of the released aortic-valve cusp guide.

67. The valve-delivery-assist tool according to claim 61, wherein the valve-delivery-assist tool is configured such that disposition of the released aortic-valve cusp guide within a non-coronary cusp of the native aortic valve and rotationally centering the aortic-valve cusp guide within the non-coronary cusp, axially disposes the released landing-support stabilizer at a predetermined height below the native aortic valve.

68. The valve-delivery-assist tool according to claim 67, wherein the predetermined height is between 2 and 10 mm.

69. The valve-delivery-assist tool according to claim 61, wherein the valve-delivery-assist tool is configured such that after lateral deflection of the aortic-valve cusp guide and expansion of the landing-support stabilizer, the released landing-support stabilizer and the released aortic-valve cusp guide are rotationally stationary with respect to one another.

70. The valve-delivery-assist tool according to claim 61, wherein the landing-support stabilizer comprises an elongate slender rod.

71. The valve-delivery-assist tool according to claim 70, wherein the elongate slender rod of the released landing-support stabilizer is shaped as a partial loop or a complete loop having one or more turns.

72. The valve-delivery-assist tool according to claim 71,
wherein the valve-delivery-assist tool further comprises (a) an elongate landing-support-stabilizer shaft, which is fixed to and supports the landing-support stabilizer, and (b) an elongate support shaft, and
wherein a distal end of the elongate slender rod is fixed to the elongate support shaft such that:
disposal of the elongate support shaft in a more distal position stretches the elongate slender rod and radially compresses the elongate slender rod against the elongate support shaft, and
disposal of the elongate support shaft in a more proximal position causes the elongate slender rod to bulge away from, and radially expand with respect to, the elongate support shaft.

73. The valve-delivery-assist tool according to claim 71,
wherein the valve-delivery-assist tool further comprises (a) an elongate landing-support-stabilizer shaft, which is fixed to and supports the landing-support stabilizer, and (b) an elongate support shaft, which is shaped so as to define a channel having a distal opening through which the elongate slender rod is slidable, and
wherein the valve-delivery-assist tool is configured such that:
withdrawal of the elongate slender rod into the channel radially compresses the elongate slender rod within the elongate support shaft, and
extension of the elongate slender rod from the channel causes the elongate slender rod to bulge away from, and radially expand with respect to, the elongate support shaft.

74. The valve-delivery-assist tool according to claim 61, further comprising an elongate valve-delivery-assist support, which links together the landing-support stabilizer and the aortic-valve cusp guide.

75. The valve-delivery-assist tool according to claim 74, wherein the released landing-support stabilizer extends laterally from the elongate valve-delivery-assist support.

76. The valve-delivery-assist tool according to claim 75, wherein a best-fit plane defined by the released landing-support stabilizer forms an angle of between 45 and 90 degrees with a central longitudinal axis of the elongate valve-delivery-assist support that is straight and passes through the distal end of the elongate valve-delivery-assist support.

77. The valve-delivery-assist tool according to claim 74, wherein the elongate valve-delivery-assist support comprises:
an elongate aortic-valve cusp-guide shaft, which is fixed to and supports the aortic-valve cusp guide; and
an elongate landing-support-stabilizer shaft, which is fixed to and supports the landing-support stabilizer,
wherein the elongate aortic-valve cusp-guide shaft and the elongate landing-support-stabilizer shaft are fixed together at least partially alongside one another.

78. The valve-delivery-assist tool according to any one of claims 61-77, for use with a guidewire,
wherein the valve-delivery-assist tool further comprises a nose cone, which is shaped so as to define (a) a wider proximal base that is removably coupled to a distal end of the delivery catheter, and (b) a narrower elongate distal safety tip that extends distally from the proximal base,
wherein the nose cone is shaped so as to define a guidewire channel through the proximal base and the elongate distal safety tip, and
wherein the elongate distal safety tip is configured to assume a straight shape when the guidewire is disposed in the guidewire channel, and a curved shape when the guidewire is not disposed in the guidewire channel.

79. The valve-delivery-assist tool according to claim 78, wherein the curved shape is a pigtail shape.

80. The valve-delivery-assist tool according to any one of claims 61-77, further comprising a safety tip, which is releasably disposed compressed in the delivery catheter, configured to be released from the distal end of the delivery catheter, and shaped so as to define a guidewire channel through the safety tip.

81. The valve-delivery-assist tool according to claim 80, wherein the safety tip is configured to expand upon released from the distal end of the delivery catheter.

82. The valve-delivery-assist tool according to claim 80, wherein the safety tip comprises an elongate slender rod.

83. The valve-delivery-assist tool according to claim 82, wherein the elongate slender rod of the released safety tip is configured to be shaped as a pigtail.
